(19) United States
(12) Patent Application Publication
(10) Pub. No.: US 2019/0142966 A1
(43) Pub. Date: May 16, 2019

(54) Functionalized Nanoparticles and Compositions for Cancer Treatment and Methods

(71) Applicant: Massachusetts Institute of Technology, Cambridge, MA (US)

(72) Inventors: Natalie Artzi, Brookline, MA (US); Joao Conde, Cambridge, MA (US); Nuria Oliva, Somerville, MA (US)

(21) Appl. No.: 16/300,135

(22) PCT Filed: May 11, 2017
(86) PCT No.: PCT/US2017/032192

§ 371 Date: Nov. 9, 2018

Related U.S. Application Data
(60) Provisional application No. 62/334,538, filed on May 11, 2016.

(51) Int. Cl. A61K 47/69; C12N 15/113; A61K 31/713; A61K 41/00; A61P 35/00; A61K 47/60; A61K 47/68; A61K 47/62; A61K 39/44; A61M 5/19; A61N 5/06

(52) U.S. Cl. CPC A61K 47/6929; C12N 2320/32; A61K 31/713; A61K 47/6923; A61K 41/0042; A61P 35/00; A61K 47/60; A61K 47/6845; A61K 47/62; A61K 39/44; A61K 47/6903; A61M 5/19; A61N 5/062; C12N 2310/14; C12N 2310/3513; C12N 15/1135

(57) ABSTRACT

Provided herein are functionalized nanoparticles and compositions containing functionalized nanoparticles. The functionalized nanoparticles include a first metal nanoparticle functionalized with a drug and a targeting biomolecule, and a second metal nanoparticle functionalized with an siRNA and a targeting biomolecule. The compositions include the first metal nanoparticle and the second metal nanoparticle. Kits and methods of drug delivery also are provided.

### CROSS-REFERENCE TO RELATED APPLICATIONS

This application claims priority to U.S. Provisional Patent Application No. 62/334,538, filed May 11, 2016, which is incorporated herein by reference.

### BACKGROUND

Cancer is among the leading causes of mortality, with more than 10 million new cases reported every year in Europe and the U.S.A. Particularly, colorectal cancer (CRC) is the second most common cause of cancer death among men and women in Europe according to the World Health Organization, and the third most common cancer found in the U.S.A. according to the American Cancer Society and the National Cancer Institute. After breast cancer, CRC is the second most common cancer in women, and, after lung and prostate cancer, the third most common in men.

Surgery, referred to as colectomy or a segmental resection, is often the main treatment for early and late stage colorectal cancer. A reasonable proportion of colorectal cancer patients remain clinically remissive for months or years following tumor resection and chemotherapy. However, after this stage of remission, tumors recur in 30-50% of all cases, generally via metastasis. Moreover, surgery is fraught with many complications including bleeding at the surgical site, formation of blood clots in the legs, damage to nearby organs, or a combination thereof. Leakage from the anastomotic site is another complication that may lead to infection, thereby delaying further treatment. Compromised healing following surgery also may lead to scar tissue and bowel obstruction along with tissue adhesions.

Conventional cancer therapy involves the use of anti-neoplastic agents that typically do not significantly distinguish cancer cells and normal cells, and/or eliminate the risk of cancer recurrence. Previous therapies for CRC also have not provided sufficient specificity and structural guidance to promote (i) tumor inhibition after remission, and/or (ii) the full regeneration of the injured intestinal tissue after surgery.

There remains a need for compositions having (i) the ability to distinguish cancer cells and normal cells, (ii) improved antitumor efficacy, (iii) the ability to eliminate or at least significantly reduce the risk of cancer recurrence, (iv) the ability to provide a multi-modal approach to cancer treatment, or (v) a combination thereof.

### BRIEF SUMMARY

Provided herein are metal nanoparticles and compositions that may be used to treat and/or reduce the recurrence of diseased cells, such as cancer cells, selectively, effectively, in a multi-modal manner, or a combination thereof.

In one aspect, a first metal nanoparticle is provided, which is functionalized with a drug and a targeting biomolecule. The targeting biomolecule may be configured to bind to a unique or overexpressed biomarker of a diseased cell, such as a cancer cell. The drug that is conjugated to the first metal nanoparticle may be an anti-angiogenic agent. The first metal nanoparticle also may be a nanorod, including a gold nanorod. The first metal nanoparticle may be configured to release heat and/or the drug upon exposure to one or more wavelengths of light, such as near-infrared light. The first metal nanoparticle may be dispersed in a medium, such as a hydrogel.

In another aspect, a second metal nanoparticle is provided, which is functionalized with a targeting biomolecule and a small interfering RNA (siRNA). The siRNA may be configured to silence a gene of a diseased cell. The siRNA may include an anti-Kras siRNA. The targeting biomolecule may be configured to bind to a unique or overexpressed biomarker of a diseased cell, such as a cancer cell. The second metal nanoparticle also may be functionalized with a fusogenic peptide. The second metal nanoparticle also may be a nanosphere, such as a gold nanosphere. The second metal nanoparticle may be dispersed in a medium, such as a hydrogel.

In a further aspect, compositions are provided that include a first metal nanoparticle and a second metal nanoparticle. The first metal nanoparticle may be functionalized with a drug and a targeting biomolecule. The second metal nanoparticle may be functionalized with a targeting biomolecule and an siRNA. A fusogenic peptide also may be conjugated to the second metal nanoparticle. The compositions may be dispersed in a medium, such as a hydrogel.

In a still further aspect, methods of drug delivery are provided. The methods, in embodiments, include providing a first solution that includes a first polymer component comprising a first polymer having one or more aldehydes; providing a second solution that includes at least one of (i) a dendrimer comprising at least two branches with one or more surface groups, wherein about 25% to 100% of the surface groups have at least one primary or secondary amine, and (ii) a second polymer component that includes a second polymer having one or more amines; combining the first and second solutions together to produce a hydrogel composite; and contacting one or more biological tissues with the hydrogel composite, wherein at least one of the first solution and the second solution includes a composition as provided herein or a component thereof. The methods also may include irradiating the first metal nanoparticle with a light source, wherein the light source emits one or more wavelengths of light that are (i) converted to heat by the first metal nanoparticle, (ii) effective to release the drug from the first metal nanoparticle, or (iii) a combination thereof.

In yet another aspect, kits for making a hydrogel composite are provided. The kit may include a first part that includes a first solution, and a second part that includes a second solution. The first solution may include a first polymer component that includes a first polymer having one or more aldehydes. The second solution may include at least one of (i) a dendrimer having at least two branches with one or more surface groups, wherein about 25% to 100% of the surface groups include at least one primary or secondary amine, and (ii) a second polymer component including a second polymer having one or more amines. At least one of the first solution and the second solution includes a composition as provided herein or a component thereof.

In a further aspect, methods for local delivery of a drug to a biological tissue are provided. The methods may include applying to the biological tissue a hydrogel in which a first metal nanoparticle and/or a second metal nanoparticle has been dispersed, and permitting the first metal nanoparticle and/or a second metal nanoparticle to diffuse from the composition into the biological tissue. The methods of local drug delivery also may include irradiating the first metal nanoparticle with a light source, wherein the light source emits one or more wavelengths of light effective to release a drug from the first metal nanoparticle.

### BRIEF DESCRIPTION OF THE DRAWINGS

FIG. 1 depicts one embodiment of a kit containing the components of a hydrogel composite or composition.

FIG. 2 depicts the percentage of Avastin® released from one embodiment of a functionalized metal nanoparticle.

FIG. 3A depicts the VEGF expression in colorectal cancer cells following treatment with Avastin® antibody previously incubated with increasing temperatures for 2 minutes (the same time as the near infra-red irradiation time that produced near 100% Avastin® release from an embodiment of the first metal nanoparticle).

FIG. 3B depicts the VEGF expression in colorectal cancer cells following treatment with Avastin® released from an embodiment of the first metal nanoparticle previously irradiated for increasing exposure times to a NIR laser.

FIG. 4A depicts a quantification of the uptake of one embodiment of nanoparticles by 3T3 fibroblasts.

FIG. 4B depicts a quantification of the uptake of one embodiment of nanoparticles by LoVo-Luc colorectal cancer cells.

FIG. 5 depicts Kras expression in LoVo-Luc colorectal cancer cells at 24, 48, and 72 hours of incubation with 1 and 10 nM of embodiments of siRNA gold nanospheres functionalized with HA1 peptide, and embodiments that lack an HA1 peptide.

FIG. 6A depicts VEGF expression in colorectal cancer cells following treatment with several embodiments of gold nanorods.

FIG. 6B depicts cell viability following treatment with several embodiments of gold nanorods.

FIG. 7A depicts luciferase activity as a measure for the tumor burden following treatment with an embodiment of a composition after tumor resection (n=5, statistical analysis performed using a two-way analysis of variance ANOVA, ***, P<0.001).

FIG. 7B depicts luciferase activity as a measure for the tumor burden following treatment with an embodiment of a composition prior to tumor resection (n=5, statistical analysis performed using a two-way analysis of variance ANOVA, ***, P<0.001).

FIG. 8 depicts luciferase activity as a measure for the tumor burden of mice treated with an embodiment of gene therapy (siRNA gold nanospheres), an embodiment of chemo-therapy (drug gold nanorods), an embodiment of phototherapy (gold nanorods), an embodiment of double therapy (chemo+gene, gene+photo, chemo+photo), and an embodiment of triple therapy (gene, chemo and photo therapy combination)(n=5, statistical analysis performed with a two-way ANOVA, ***, P<0.001).

FIG. 9A depicts a quantification of the signal from an embodiment of nanorods following the application of single, dual, and triple therapy combinations in mice using a live imaging system at day 15.

FIG. 9B depicts a quantification of the signal from an embodiment of nanospheres following the application of single, dual, and triple therapy combinations in mice using a live imaging system at day 15.

FIG. 10 depicts Kaplan-Meier curves for mice treated with embodiments of compositions for gene therapy (siRNA gold nanospheres), chemo-therapy (drug gold nanorods), phototherapy (gold nanorods), or triple therapy (gene, chemo and photo therapy combination).

### DETAILED DESCRIPTION

Improved metal nanoparticles and compositions are provided herein that may treat one or more diseased cells with gene therapy, drug therapy, phototherapy, or a combination thereof. In embodiments, the application of a “triple therapy” that includes gene therapy, drug therapy, and phototherapy, can synergistically abrogate tumors, including colorectal tumors, and prevent their recurrence, with or without tumor resection. The methods of drug delivery provided herein can be adapted to numerous cancer cell types and to molecular targets associated with disease progression.

Drug therapy, phototherapy, or a combination thereof may be provided, as described herein, with a first metal nanoparticle that is functionalized with a drug and a targeting biomolecule. Gene therapy may be provided by a second metal nanoparticle functionalized with a target biomolecule and an siRNA. Gene therapy, drug therapy, and phototherapy may be provided by a composition including the first metal nanoparticle and the second metal nanoparticle. The first metal nanoparticle, the second metal nanoparticle, or the composition may be disposed in a medium, such as a hydrogel.

### First Metal Nanoparticle

In embodiments, the first metal nanoparticle provided herein is functionalized with a drug and a targeting biomolecule. Due to the fact that a drug is conjugated to the first metal nanoparticle, the first nanoparticle may provide drug therapy as described herein. Generally, any ratio of drug to targeting biomolecule may be conjugated to the first metal nanoparticle. In embodiments, the mol ratio of drug to targeting biomolecule conjugated to the first metal nanoparticle is about 1:4 to about 1:1.

In embodiments, the drug is present at an amount of about 10 mols to about 20 mols, or about 13 mols to about 17 mols of drug per particle of the first metal nanoparticle.

In embodiments, the targeting biomolecule is present at an amount of about 20 mols to about 40 mols, or about 28 mols to about 32 mols of targeting biomolecule per particle of the first metal nanoparticle.

In embodiments, the drug is present at an amount of about 10 mols to about 20 mols, or about 13 mols to about 17 mols of drug per particle of the first metal nanoparticle, and the targeting biomolecule is present at an amount of about 20 mols to about 40 mols of targeting biomolecule per particle of the first metal nanoparticle.

In embodiments, the drug is present at an amount of about 10 mols to about 20 mols, or about 13 mols to about 17 mols of drug per particle of the first metal nanoparticle, and the targeting biomolecule is present at an amount of about 28 mols to about 32 mols of targeting biomolecule per particle of the first metal nanoparticle.

The first metal nanoparticle may be formed of any biocompatible metal or mixture of metals. The phrase “metal nanoparticle,” as used herein, refers to a particle having [1] an average diameter of about 1 nm to about 100 nm, and [2] a structure that includes at least 95%, by weight, of one or more metals. In one embodiment, the first metal nanoparticle is a gold nanoparticle. The phrases “gold nanoparticle” or “gold nanoparticles” as used herein, refer to a particle or particles comprising gold in at least an amount of 50% by weight, and have an average diameter of about 1 nm to about 100 nm. In one embodiment, the gold nanoparticles include gold in an amount of at least 95% by weight. In another embodiment, the gold nanoparticles include gold in an amount of at least 99% by weight. In some embodiments, the uptake, in vivo biodistribution, or a combination thereof, may be controlled, at least in part, by selecting a particular size or sizes of a first metal nanoparticle.

When the first metal nanoparticle is a gold nanoparticle, the gold nanoparticle may be selected from those that are commercially available, or made by techniques known in the art, such as the citrate reduction method, e.g., see Lee, P. C. et al., J. Phys. Chem. 1982, 86(17), 3391-3395. When the citrate reduction method is used, the gold nanoparticles may include citrate groups on at least a portion of their surfaces. The citrate groups may be relied upon, at least in part, to functionalize the gold nanoparticles with a drug and a targeting biomolecule to form the first metal nanoparticles provided herein, or with a targeting biomolecule, an siRNA, and optionally a fusogenic peptide to form the second metal nanoparticle provided herein. It is well-known, for example, that a thiol functional group can undergo an exchange with a citrate group on the surface of a gold nanoparticle.

The first metal nanoparticle generally may have any shape or combination of two or more different shapes. For example, the first metal nanoparticles may be in the shape of a nanosphere, a nanorod, or a combination thereof.

The first metal nanoparticle may have a shape, or a combination of two or more different shapes having the capacity to convert one or more wavelengths of light to heat, and/or release a drug when exposed to one or more particular wavelengths of light. This feature may be achieved by imparting the first metal nanoparticle with a shape that has an absorbance peak in a particular region, such as the NIR region, so that upon exposure to light of one or more wavelengths at or near the absorbance peak, the first metal nanoparticle converts the light into heat. The heat released by the first metal nanoparticle may [1] kill and/or damage one or more diseased cells, and/or [2] facilitate release of the drug from the first metal nanoparticle. When the first metal nanoparticle is capable of converting one or more wavelengths of light to heat, the first metal nanoparticle may be used to provide phototherapy as described herein. In embodiments, the second metal nanoparticle does not have an absorbance peak at or near the absorbance peak of the first metal nanoparticle, thereby preventing the second metal nanoparticle from converting into heat the one or more wavelengths of light that are at or near the absorbance peak of the first metal nanoparticle.

In one embodiment, the first metal nanoparticle is or includes a nanorod, which is capable of converting one or more wavelengths of light to heat. A “nanorod” is a nanoparticle having an aspect ratio of at least 3:2. The nanorod may have an aspect ratio of about 3:2 to about 10:1. For example, a nanorod may have an average length of about 30 nm to about 50 nm, and an average diameter of about 5 nm to about 20 nm. In one embodiment, the first metal nanoparticle is a nanorod having an average length of about 40 nm, and an average diameter of about 10 nm. In a particular embodiment, the first metal nanoparticle includes a gold nanoparticle that is in the shape of a rod, which is referred to herein as a gold nanorod. The gold nanorod may have [1] an average length of about 30 nm to about 50 nm, and an average diameter of about 5 nm to about 20 nm; or [2] an average length of about 40 nm, and an average diameter of about 10 nm. The “nanorod” may have a shape that is at least substantially cylindrical, but the use of the phrase “average diameter” is not intended to imply that the nanorod always is at least substantially circular when viewed in cross-section at a point along its length. When the nanorod is not at least substantially circular when viewed in cross-section, the “average diameter” refers to the average largest dimension of the nanorod when viewed in cross-section at a point along its length.

The first metal nanoparticle also may include a drug linker. A “drug linker” generally is any molecule that is covalently bonded to the first metal nanoparticle and the drug. The drug linker, in embodiments, includes (i) a sulfur atom covalently bonded to the first metal nanoparticle, and (ii) an ester moiety covalently bonded to the drug. The first metal nanoparticle may include one or more types of drug linker. In one embodiment, the drug linker is a thiol-PEG-COOH drug linker, which has the following structure when the first metal nanoparticle is conjugated to a drug:

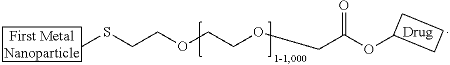

The thiol-PEG-COOH drug linker also may include one or more functional groups and/or moieties, including, but not limited to, an amide, a methylene group, an ether, an ester, or a combination thereof. For example, the thiol-PEG-COOH drug linker may include an amide and one or more methylene moieties. In one embodiment, the thiol-PEG-COOH drug linker includes an amide and five methylene moieties, and has the following structure: HS—C₂H₄—CONH-PEG-O—C₃H₆—COOH. The thiol-PEG-COOH drug linker may have a weight average molecule weight of about 2,000 g/mol to about 5,000 g/mol, about 3,000 g/mol to about 4,000 g/mol, or about 3,500 g/mol.

The drug that is conjugated to a first metal nanoparticle may be active, i.e., exhibit a therapeutic effect, whether or not it remains covalently bonded to the first metal nanoparticle and/or the drug linker. A drug may be configured to [1] remain conjugated to a first metal nanoparticle, [2] be released from a first metal nanoparticle, or [3] a combination thereof. The phrase “released from a first meal nanoparticle,” as used herein, refers to the severing of one or more covalent bonds that conjugate the drug to the first metal nanoparticle, either directly or through a drug linker. For example, a drug may be released from a first metal nanoparticle upon the breaking of a covalent bond that [1] connects the first metal nanoparticle to the drug linker, [2] connects the first metal nanoparticle to the drug, [3] connects the drug linker to the drug, [4] exists between two or more atoms of the drug linker, or [5] a combination thereof. In one embodiment, a drug remains covalently bonded to the drug linker, or at least a portion thereof, upon the release of the drug from the first metal nanoparticle. In another embodiment, a covalent bond connecting the drug and drug linker is severed upon release of the drug from the first metal nanoparticle.

The first metal nanoparticle may be dispersed in a medium. The medium may be any medium with which the first metal nanoparticle is compatible, including media that aid in the handling and/or delivery of a first metal nanoparticle. In one embodiment, the first metal nanoparticle is dispersed in a hydrogel. The hydrogel may include the contact product of the first solution and the second solution described herein.

### Second Metal Nanoparticle

In embodiments, the second metal nanoparticle provided herein is functionalized with an siRNA and a targeting biomolecule. Due to the fact that an siRNA is conjugated to the second metal nanoparticle, the second metal nanoparticle may be used to provide gene therapy as described herein. Generally, any ratio of siRNA to targeting biomolecule may be conjugated to the second metal nanoparticle. In embodiments, the mol ratio of siRNA to targeting biomolecule conjugated to the second metal nanoparticle is about 6:1 to about 2:1.

In embodiments, the siRNA is present at an amount of about 90 mols to about 120 mols, or about 100 mols to about 110 mols of siRNA per particle of the second metal nanoparticle.

In embodiments, the targeting biomolecule is present at an amount of about 20 mols to about 40 mols, or about 28 mols to about 32 mols of targeting biomolecule per particle of the second metal nanoparticle.

In embodiments, the siRNA is present at an amount of about 90 mols to about 120 mols, or about 100 mols to about 110 mols of siRNA per particle of the second metal nanoparticle, and the targeting biomolecule is present at an amount of about 20 mols to about 40 mols of targeting biomolecule per particle of the second metal nanoparticle.

In embodiments, the siRNA is present at an amount of about 90 mols to about 120 mols, or about 100 mols to about 110 mols of siRNA per particle of the second metal nanoparticle, and the targeting biomolecule is present at an amount of about 28 mols to about 32 mols of targeting biomolecule per particle of the second metal nanoparticle.

In embodiments, the second metal nanoparticle is functionalized with an siRNA, a targeting biomolecule, and a fusogenic peptide. Generally, any ratio of siRNA to targeting biomolecule to fusogenic peptide may be conjugated to the second metal nanoparticle. In embodiments, the mol ratio of siRNA to targeting biomolecule to fusogenic peptide conjugated to the second metal nanoparticle is about 6:1:1 to about 2:1:1.

In embodiments, the fusogenic peptide is present at an amount of about 20 mols to about 40 mols per particle of the second metal nanoparticle, or about 28 mols to about 32 mols per particle of the second metal nanoparticle.

In embodiments, the siRNA is present at an amount of about 90 mols to about 120 mols, or about 100 mols to about 110 mols of siRNA per particle of the second metal nanoparticle, the targeting biomolecule is present at an amount of about 20 mols to about 40 mols of targeting biomolecule per particle of the second metal nanoparticle, and the fusogenic peptide is present at an amount of about 20 mols to about 40 mols per particle of the second metal nanoparticle.

In embodiments, the siRNA is present at an amount of about 90 mols to about 120 mols, or about 100 mols to about 110 mols of siRNA per particle of the second metal nanoparticle, the targeting biomolecule is present at an amount of about 20 mols to about 40 mols of targeting biomolecule per particle of the second metal nanoparticle, and the fusogenic peptide is present at an amount of about 28 mols to about 32 mols per particle of the second metal nanoparticle.

In embodiments, the siRNA is present at an amount of about 90 mols to about 120 mols, or about 100 mols to about 110 mols of siRNA per particle of the second metal nanoparticle, the targeting biomolecule is present at an amount of about 28 mols to about 32 mols of targeting biomolecule per particle of the second metal nanoparticle, and the fusogenic peptide is present at an amount of about 20 mols to about 40 mols per particle of the second metal nanoparticle.

In embodiments, the siRNA is present at an amount of about 90 mols to about 120 mols, or about 100 mols to about 110 mols of siRNA per particle of the second metal nanoparticle, the targeting biomolecule is present at an amount of about 28 mols to about 32 mols of targeting biomolecule per particle of the second metal nanoparticle, and the fusogenic peptide is present at an amount of about 28 mols to about 32 mols per particle of the second metal nanoparticle.

The second metal nanoparticle may be formed of any biocompatible metal or mixture of metals. In one embodiment, the second metal nanoparticle is a gold nanoparticle. When the second metal nanoparticle is a gold nanoparticle, the gold nanoparticle may be selected from those that are commercially available, or made by techniques known in the art.

The second metal nanoparticle generally may have any shape or combination of two or more different shapes. For example, the second metal nanoparticles may be in the shape of a sphere, a rod, or a combination thereof. In one embodiment, the second metal nanoparticle is a nanosphere. The term “nanosphere,” as used herein, refers to nanoparticles having at least a substantially spherical shape, and an average diameter of about 1 nm to about 100 nm.

In embodiments, the average diameter of the second metal nanoparticle is about 5 nm to about 50 nm, about 5 nm to about 40 nm, about 5 nm to about 35 nm, about 15 nm to about 35 nm, about 25 nm to about 35 nm, or about 30 nm. The average diameter of the nanoparticles herein may be determined by transmission electron microscopy (TEM) images. The use of the phrase “average diameter” should not be construed as implying that the second metal nanoparticle is necessarily spherical in shape. When the second metal nanoparticle is not at least substantially spherical in shape, the “average diameter” refers to the average largest dimension of the second metal nanoparticle.

In one embodiment, the second metal nanoparticle is a gold nanoparticle having an average diameter of about 5 nm to about 50 nm, about 5 nm to about 40 nm, about 5 nm to about 35 nm, about 15 nm to about 35 nm, about 25 nm to about 35 nm, or about 30 nm.

In one embodiment, the second metal nanoparticle is a gold nanosphere having an average diameter of about 5 nm to about 50 nm, about 5 nm to about 40 nm, about 5 nm to about 35 nm, about 15 nm to about 35 nm, about 25 nm to about 35 nm, or about 30 nm.

The second metal nanoparticle may be dispersed in a medium. The medium may be any medium with which the second metal nanoparticle is compatible, including media that aid in the handling and/or delivery of a second metal nanoparticle. In one embodiment, the second metal nanoparticle is dispersed in a hydrogel. The hydrogel may include the contact product of the first solution and the second solution described herein.

### Compositions

Also provided herein are compositions that include a first metal nanoparticle and a second metal nanoparticle. The compositions provided herein may be dispersed in a medium. The medium may be any medium with which the first metal nanoparticle and the second metal nanoparticle are compatible, including media that aid in the handling and/or delivery of the first metal nanoparticle and the second metal nanoparticle. In one embodiment, the compositions further comprise a hydrogel in which the first metal nanoparticle and the second metal nanoparticle are dispersed. The hydrogel may include the contact product of the first solution and the second solution described herein. The first metal nanoparticle and the second metal nanoparticle may be substantially evenly dispersed in the hydrogel. The first metal nanoparticle and the second metal nanoparticle may be substantially evenly and unevenly dispersed, respectively, in the hydrogel, or vice versa.

### siRNA

The siRNA generally may include one or more types of siRNA capable of silencing one or more genes, including protein coding genes, of a diseased cell. For example, the siRNA may be capable of silencing an oncogene driver in tumor progression. An siRNA may be selected based on its ability to silence one or more genes that are known to exist in a diseased cell, such as a tumor cell. An siRNA may “silence” a gene when the siRNA interferes with or “knocks down” gene expression. This may be achieved with complementary nucleotide sequences by degrading mRNA after transcription, which results in no translation.

In one embodiment, the siRNA is an anti-Kras siRNA, which silences an oncogene driver in colorectal cancer progression, known as Kras (Kirsten Rat Sarcoma Viral Oncogene Homolog).

The siRNA may include a thiol moiety. When the siRNA includes a thiol moiety, the siRNA may be conjugated to the second metal nanoparticle via a sulfide bond. The thiol moiety may be located at a terminal position of the siRNA.

### Fusogenic Peptide

The fusogenic peptide generally may be any one or more types of peptides capable of at least partially destabilizing the endosomal membrane, which may stimulate endosomal discharge. The endosomal discharge may occur by a pH-responsive mechanism. Not wishing to be bound by any particular theory, it is believed that the fusogenic peptide may enhance siRNA uptake by a diseased cell.

In one embodiment, the fusogenic peptide includes an HA1 peptide (Influenza Hemagglutinin—N-YPYDVPDYA-C). In other embodiments, the fusogenic peptide includes an HA1 peptide, B18, diINF-7, or a combination thereof. B18 is an 18-residue fusogenic peptide from the sea urchin fertilization protein. diINF-7 is an influenza-derived fusogenic peptide.

### Targeting Biomolecule

The first metal nanoparticle, the second metal nanoparticle, or a combination thereof may be functionalized with a targeting biomolecule that is configured to bind to a receptor that is unique and/or overexpressed by a diseased cell, such as a cancer cell. The targeting biomolecule, therefore, may result in the selective cellular uptake of the compositions provided herein or a component thereof in the target diseased cells. The targeting biomolecule, upon cellular uptake, also may result in receptor-mediated endocytosis (RME). The targeting biomolecules may generally include any targeting biomolecule, such as a peptide, that is configured to bind to one or more receptors of diseased cells, such as receptors that are unique to diseased cells, upregulated by diseased cells, or a combination thereof.

For example, the targeting biomolecule may include a peptide that targets cancer cells of a particular type. In one embodiment, the targeting biomolecule is a TCP-1 peptide (N-CTPSPFSHC-C), which targets colorectal cancer cells.

As a further example, epidermal growth factor receptor (EGFR) expression is upregulated in multiple cancer types, such as breast, head, neck, cervical, ovarian, bladder, esophageal, endometrial, lung, and colorectal cancer. The upregulation of EGFR may be correlated with increased recurrence-rate and/or reduced overall survival. Therefore, in one embodiment, the targeting biomolecule is an EGF mimicking peptide (EGFmp) that is capable of binding to EGFR.

Since other targeting biomolecules may be used, the first metal nanoparticle and/or the second metal nanoparticle may be used to deliver [1] photo and/or drug therapy and/or [2] gene therapy, respectively, to many types of cancer cells by conjugation of other targeting biomolecules, such as growth factor mimicking peptides, to target other commonly overexpressed receptors in cancer cells, such as FGF2R, VEGFR, and PDGFR.

The targeting biomolecule may be selected from an EFG mimicking peptide, an FGF2 mimicking peptide, a VEGF mimicking peptide, a PDGF mimicking peptide, or a combination thereof. These binding peptides are configured to bind, respectively, to the following receptors: EGFR, FGF2R, and PDGFR.

The first metal nanoparticle and/or the second metal nanoparticle may be functionalized with one type of targeting biomolecules, or two or more types of targeting biomolecules. The first metal nanoparticle may be functionalized with a first type of targeting biomolecule, and the second metal nanoparticle may be functionalized with a second type of targeting biomolecule. The first metal nanoparticle and the second metal nanoparticle may be functionalized with the same type of targeting biomolecule(s). The targeting biomolecule may include one type of binding peptide, or two or more types of binding peptide. Each type of binding peptide may be configured to bind to a different receptor.

When the targeting biomolecule is or includes a binding peptide, the binding peptides may be synthetic peptides. Not wishing to be bound by any particular theory, it is believed that synthetic peptides may result in higher stability when the first metal nanoparticle and/or second metal nanoparticle is in solution, and/or higher and more selective uptake. It also is believed that synthetic peptides may interact with growth factor receptors, and possibly elicit RME without activating the downstream signaling pathway.

The targeting biomolecule may be conjugated to the first metal nanoparticle, the second metal nanoparticle, or a combination thereof through a targeting biomolecule linker. The targeting biomolecule linker generally may be any molecule capable of covalently bonding to the targeting biomolecule and at least one of the first metal nanoparticle and the second metal nanoparticle. In one embodiment, the targeting biomolecule linker includes (i) a sulfur atom that is covalently bonded to at least one of the first metal nanoparticle and the second metal nanoparticle, and (ii) an ester moiety covalently bonded to the targeting biomolecule.

In embodiments, the targeting biomolecule linker is a thiol-PEG-COOH targeting biomolecule linker, which may have the following structure when the first and/or second metal nanoparticle is conjugated to a targeting biomolecule:

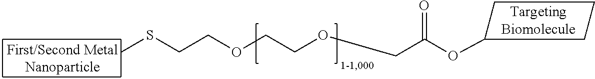

The thiol-PEG-COOH targeting biomolecule linker also may include one or more functional groups and/or moieties, including, but not limited to, an amide, a methylene group, an ether, an ester, or a combination thereof. For example, the thiol-PEG-COOH targeting biomolecule linker may include an amide and one or more methylene moieties. In one embodiment, the thiol-PEG-COOH targeting biomolecule linker includes an amide and five methylene moieties, and has the following structure: HS—C₂H₄—CONH-PEG-O—C₃H₆—COOH. The thiol-PEG-COOH targeting biomolecule linker may have a weight average molecule weight of about 2,000 g/mol to about 5,000 g/mol, about 3,000 g/mol to about 4,000 g/mol, or about 3,500 g/mol.

### Drugs

Generally, any drug may be conjugated to a first metal nanoparticle. The drug that is conjugated to a first metal nanoparticle may include a single type of drug or two or more types of drug. Since the response to drugs can vary from patient to patient, the first metal nanoparticle provided herein may be personalized on a patient-by-patient basis.

In embodiments, the drug may include one or more anti-angiogenic agents, one or more chemotherapeutic agents, or a combination thereof.

In embodiments, the drug includes an anti-angiogenic agent. An “anti-angiogenic agent” includes drugs that inhibit the growth of blood vessels. In one embodiment, the anti-angiogenic agent is bevacizumab (Avastin®). Other anti-angiogenic agents that may be conjugated to the first metal nanoparticles provided herein include, but are not limited to, axitinib, cabozantinib, cetuximab, everolimus, lenalidomide, pazopanib, ramucirumab, regorafenib, sorafenib, sunitinib, thalidomide, vandetanib, and ziv-aflibercept.

In embodiments, the drug conjugated to a first metal nanoparticle includes one or more chemotherapeutic agents. A “chemotherapeutic agent” is a chemical compound useful in the treatment of cancer. Examples of chemotherapeutic agents include, but are not limited to, alkylating agents such as thiotepa and cyclosphosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrirnidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxanes, e.g. paclitaxel (TAXOL®, Bristol-Myers Squibb Oncology, Princeton, N.J.) and docetaxel (TAXOTERE®, Rhône-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; esperamicins; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

In one embodiment, the one or more chemotherapeutic agents includes doxorubicin.

### Hydrogels

The first metal nanoparticle, the second metal nanoparticle, or a combination thereof may be dispersed in a hydrogel. The first metal nanoparticle and/or the second metal nanoparticle may be dispersed at least substantially evenly in a hydrogel, or unevenly in a hydrogel.

When a composition that includes the first metal nanoparticle and the second metal nanoparticle is dispersed in a hydrogel, the first metal nanoparticle may be dispersed evenly in the hydrogel, and the second metal nanoparticle may be dispersed unevenly in the hydrogel, or vice versa. The concentrations of the first metal nanoparticle and the second metal nanoparticle in a hydrogel may be the same or different. In one embodiment, the concentrations of the first metal nanoparticle and the second metal nanoparticle in a hydrogel are substantially the same. In another embodiment, the concentration of the first metal nanoparticle in a hydrogel is greater than the concentration of the second metal nanoparticle. In yet another embodiment, the concentration of the first metal nanoparticle in a hydrogel is less than the concentration of the second metal nanoparticle. The concentration of the first metal nanoparticle in a hydrogel may be about 5 nM to about 50 nM, about 5 to about 30 nM, about 5 nM to about 20 nM, about 5 nM to about 15 nM, or about 10 nM. The concentration of the second metal nanoparticle in a hydrogel may be about 5 nM to about 50 nM, about 5 to about 30 nM, about 5 nM to about 20 nM, about 5 nM to about 15 nM, or about 10 nM. In particular embodiment, the concentration of the first metal nanoparticle in a hydrogel is about 5 nM to about 10 nM, and the concentration of the second metal nanoparticle is about 5 nM to about 10 nM.

The hydrogels described herein generally may include any biocompatible hydrogel. The hydrogel may serve as a matrix material for controlled delivery of drug, localized drug delivery, sustained delivery of drug, or a combination thereof. Methods of locally delivering a drug may include applying to a biological tissue, such as a human tissue, a drug delivery composition as provided herein, and permitting at least one drug, the first metal nanoparticle, and/or the second metal nanoparticle to transport (e.g., diffuse) from the composition into the biological tissue. The hydrogel may adhere to one or more biological tissues, thereby reducing or eliminating the risk of unwanted material migration following application of the hydrogel to one or more selected tissue sites. The hydrogel generally may be degradable, injectable, or a combination thereof. At least one of the first metal nanoparticle and the second metal nanoparticle may be added to the hydrogel after hydrogel formation.

The hydrogel may be used as a treatment or prophylaxis of cancer in a patient.

The hydrogel may include a contact product of [1] a first solution that includes the first polymer component described herein, and [2] a second solution that includes the second polymer component and/or the dendrimer component described herein. In embodiments, at least one of the first solution and the second solution comprises a composition as described herein or a component thereof. The “component thereof” may include a first metal nanoparticle or a second metal nanoparticle. Therefore, [1] the first solution may include a first metal nanoparticle and a second metal nanoparticle, [2] the second solution may include a first metal nanoparticle and a second metal nanoparticle, [3] the first solution and the second solution may include a first metal nanoparticle and a second metal nanoparticle, [4] the first solution may include a first metal nanoparticle and the second solution may include a second metal nanoparticle, [5] the first solution may include a second metal nanoparticle and the second solution may include a first metal nanoparticle, [6] the first solution may include a first metal nanoparticle, and the second metal nanoparticle may be added after hydrogel formation, [7] the first solution may include a second metal nanoparticle, and the first metal nanoparticle may be added after hydrogel formation, [8] the second solution may include a first metal nanoparticle, and the second metal nanoparticle may be added after hydrogel formation, [9] the second solution may include a second metal nanoparticle, and the first metal nanoparticle may be added after hydrogel formation, or [10] the first solution and the second solution include a first metal nanoparticle or a second metal nanoparticle, and the other metal nanoparticle is added after hydrogel formation.

The first metal nanoparticle and/or the second metal nanoparticle may be added to the hydrogel after hydrogel formation.

The first metal nanoparticle and the second metal nanoparticle may be present in the first solution, the second solution, or a combination thereof in an amount sufficient to impart the resulting hydrogel with a concentration of each of the first metal particle and the second metal nanoparticle of about 5 μM to about 75 μM, about 15 μM to about 65 μM, or about 25 μM to about 50 μM. When the first metal nanoparticle and the second metal nanoparticle are present in the first solution and/or the second solution, the concentrations of the first metal nanoparticle and the second metal nanoparticle may be the same or different. For example, the first solution may include a first metal nanoparticle at a concentration that is the same or different as the concentration of the second metal nanoparticle in the first solution or the second solution.

The first metal nanoparticle and/or the second metal nanoparticle may be disposed in a solution having components with which the first metal nanoparticle and/or the second metal nanoparticle is incapable of reacting.

The rate of drug delivery may be controlled, at least in part, by imparting the first metal nanoparticle and/or the second metal nanoparticle with one or more functional groups capable of reacting with a functional group of at least one component of the hydrogel in which the first metal nanoparticle and/or the second metal nanoparticle is dispersed. If the first metal nanoparticle and/or the second metal nanoparticle does not include functional groups capable of reacting with a functional group of at least one component of the hydrogel in which the first metal nanoparticle and/or the second metal nanoparticle is dispersed, then the rate of drug delivery may be dictated by the diffusion of the first metal nanoparticle and/or the second metal nanoparticle from the hydrogel. If the first metal nanoparticle and/or the second metal nanoparticle does include a functional group capable of reacting with a functional group of at least one component of the hydrogel, then the rate of drug delivery may be dictated by the degradation rate of the hydrogel, the diffusion of the first metal nanoparticle and/or the second metal nanoparticle from the hydrogel, or a combination thereof.

When a drug is substantially inactive, i.e., exhibits substantially no therapeutic effect, until it is released from a first metal nanoparticle upon exposure of the first metal nanoparticle to one or more wavelengths of light, then the rate of drug delivery may be controlled by the duration and/or frequency of the exposure of the first metal nanoparticle to the one or more wavelengths of light. In embodiments, the rate of drug delivery may be controlled by the duration and/or frequency of the exposure of the first metal nanoparticle to one or more wavelengths of light, and the rate of the release of first metal nanoparticle from the hydrogel may be controlled, at least in part, by imparting the first metal nanoparticle with one or more functional groups capable of reacting with a functional group of at least one component of the hydrogel in which the first metal nanoparticle is dispersed. In further embodiments, the rate of drug delivery may be controlled by the duration and/or frequency of the exposure of the first metal nanoparticle to one or more wavelengths of light, and the rate of the release of first metal nanoparticle from the hydrogel may be controlled, at least in part, by diffusion of the first metal nanoparticle from the hydrogel.

Generally, the hydrogel composites and compositions, including drug delivery compositions, provided herein may be formed by combining a first solution and a second solution as described herein. The first solution and the second solution may be aqueous macromer solutions. The first solution and/or the second solution may independently include water, phosphate buffer saline (PBS), Dulbecco's Modified Eagle's Medium (DMEM), or any combination thereof.

The first solution, in embodiments, includes a composition described herein or a component thereof and a first polymer component. The first solution, in other embodiments, includes a first polymer component without a composition described herein or a component thereof.

The second solution may include at least one of a dendrimer and a second polymer component. The dendrimer and/or second polymer component generally have one or more functional groups capable of reacting with the one or more functional groups on the first polymer. The second dendrimer and/or second polymer component, in particular embodiments, include one or more amines. The second solution, in other embodiments, also includes a composition as described herein or a component thereof.

The first solution and the second solution, in embodiments, are combined to form the hydrogel composites and compositions described herein. When combined, the aldehyde groups of the first solution may react with the amines that are present in the second solution. This reaction is referred to herein as “curing” or “gelling.”

In embodiments, the first metal nanoparticle and/or the second metal nanoparticle is substantially evenly (i.e., uniformly) dispersed in the first solution. In other embodiments, the first metal nanoparticle and/or the second metal nanoparticle is substantially evenly dispersed in the first solution and the second solution. In further embodiments, the first metal nanoparticle and/or the second metal nanoparticle is evenly dispersed in the second solution. Although the first metal nanoparticle and/or the second metal nanoparticle is evenly dispersed in preferred embodiments, other embodiments may not have an even dispersement of the first metal nanoparticle and/or the second metal nanoparticle.

In embodiments, the concentration of the first metal nanoparticle in the first solution is about 0.01% to about 30% by weight of the first solution. In some embodiments, the concentration of the first metal nanoparticle in the first solution is about 0.01% to about 25% by weight of the first solution. In further embodiments, the concentration of the first metal nanoparticle in the first solution is about 0.01% to about 20% by weight of the first solution. In still further embodiments, the concentration of the first metal nanoparticle in the first solution is about 0.01% to about 15% by weight of the first solution.

In embodiments, the concentration of the first metal nanoparticle in the second solution is about 0.01% to about 30% by weight of the second solution. In some embodiments, the concentration of the first metal nanoparticle in the second solution is about 0.01% to about 25% by weight of the second solution. In further embodiments, the concentration of the first metal nanoparticle in the second solution is about 0.01% to about 20% by weight of the second solution. In still further embodiments, the concentration of the first metal nanoparticle in the second solution is about 0.01% to about 15% by weight of the second solution.

In embodiments, the concentration of the second metal nanoparticle in the first solution is about 0.01% to about 30% by weight of the first solution. In some embodiments, the concentration of the second metal nanoparticle in the first solution is about 0.01% to about 25% by weight of the first solution. In further embodiments, the concentration of the second metal nanoparticle in the first solution is about 0.01% to about 20% by weight of the first solution. In still further embodiments, the concentration of the second metal nanoparticle in the first solution is about 0.01% to about 15% by weight of the first solution.

In embodiments, the concentration of the second metal nanoparticle in the second solution is about 0.01% to about 30% by weight of the second solution. In some embodiments, the concentration of the second metal nanoparticle in the second solution is about 0.01% to about 25% by weight of the second solution. In further embodiments, the concentration of the second metal nanoparticle in the second solution is about 0.01% to about 20% by weight of the second solution. In still further embodiments, the concentration of the second metal nanoparticle in the second solution is about 0.01% to about 15% by weight of the second solution.

In embodiments, the concentration of the first metal nanoparticle in the hydrogel composites or compositions described herein is about 0.01% to about 10% by weight of the hydrogel composite or composition. In some embodiments, the concentration of the first metal nanoparticle in the hydrogel composites or compositions described herein is about 0.01% to about 8% by weight of the hydrogel composite or composition. In certain embodiments, the concentration of the first metal nanoparticle in the hydrogel composites or compositions described herein is about 0.01% to about 6% by weight of the hydrogel composite or composition. In particular embodiments, the concentration of the first metal nanoparticle in the hydrogel composites or compositions described herein is about 0.01% to about 5% by weight of the hydrogel composite or composition.

In embodiments, the concentration of the second metal nanoparticle in the hydrogel composites or compositions described herein is about 0.01% to about 10% by weight of the hydrogel composite or composition. In some embodiments, the concentration of the second metal nanoparticle in the hydrogel composites or compositions described herein is about 0.01% to about 8% by weight of the hydrogel composite or composition. In certain embodiments, the concentration of the second metal nanoparticle in the hydrogel composites or compositions described herein is about 0.01% to about 6% by weight of the hydrogel composite or composition. In particular embodiments, the concentration of the second metal nanoparticle in the hydrogel composites or compositions described herein is about 0.01% to about 5% by weight of the hydrogel composite or composition.

In embodiments, the concentration of first polymer component in the first solution is about 0.01% to about 40% by weight of the first solution. In further embodiments, the concentration of first polymer component in the first solution is about 0.01% to about 30% by weight of the first solution. In some embodiments, the concentration of first polymer component in the first solution is about 0.01% to about 20% by weight of the first solution. In a particular embodiment, the concentration of first polymer component in the first solution is about 20% by weight of the first solution. In additional embodiments, the concentration of first polymer component in the first solution is about 0.01% to about 10% by weight of the first solution. Typically, the concentration may be tailored and/or adjusted based on the particular application, tissue type, and/or the type and concentration of dendrimer and/or second polymer component used.

In embodiments, the concentration of the first polymer component in the hydrogel composites or compositions described herein is about 0.01% to about 20% by weight of the hydrogel composite or composition. In further embodiments, the concentration of the first polymer component in the hydrogel composites or compositions described herein is about 0.01% to about 15% by weight of the hydrogel composite or composition. In some embodiments, the concentration of the first polymer component in the hydrogel composites or compositions described herein is about 0.01% to about 10% by weight of the hydrogel composite or composition. In still further embodiments, the concentration of the first polymer component in the hydrogel composites or compositions described herein is about 0.01% to about 7% by weight of the hydrogel composite or composition.

In embodiments, the total concentration of dendrimer and second polymer component in the second solution is about 0.01% to about 40% by weight of the second solution. In further embodiments, the total concentration of dendrimer and second polymer component in the second solution is about 0.01% to about 30% by weight of the second solution. In some embodiments, the total concentration of dendrimer and second polymer component in the second solution is about 0.01% to about 20% by weight of the second solution. In additional embodiments, the total concentration of dendrimer and second polymer component in the second solution is about 0.01% to about 10% by weight of the second solution. In a particular embodiment, the total concentration of dendrimer and second polymer component in the second solution is about 25% by weight of the second solution. Typically, the concentration may be tailored and/or adjusted based on the particular application, tissue type, and/or the type and concentration of first polymer component used. As used herein, the phrase “total concentration of dendrimer and second polymer component” refers to the sum of the concentration of dendrimer and the concentration of the second polymer component. The phrase does not imply that both a dendrimer and a second polymer component must be present in the second solution. The second solution may include a dendrimer, second polymer component, or both a dendrimer and second polymer component.

In embodiments, the total concentration of dendrimer and second polymer component in the hydrogel composites or compositions described herein is about 0.01% to about 20% by weight of the hydrogel composite or composition. In further embodiments, the total concentration of dendrimer and second polymer component in the hydrogel composites or compositions described herein is about 0.01% to about 15% by weight of the hydrogel composite or composition. In some embodiments, the total concentration of dendrimer and second polymer component in the hydrogel composites or compositions described herein is about 0.01% to about 10% by weight of the hydrogel composite or composition. In still further embodiments, the total concentration of dendrimer and second polymer component in the hydrogel composites or compositions described herein is about 0.01% to about 7% by weight of the hydrogel composite or composition.

### First Polymer Component

The first polymer component generally includes a first polymer with one or more functional groups capable of reacting with one or more functional groups on a biological tissue and/or one or more functional groups on the dendrimer component and/or second polymer component of the second solution. The first polymer component, in embodiments, comprises a first polymer having one or more aldehyde groups.

The polymers of the first polymer component may be selected from any biocompatible polymers capable of forming or imparting certain characteristics to the hydrogel composites and compositions described herein. The polymers of the first polymer component, for example, may be selected from at least one polysaccharide, at least one hydrophilic polymer, at least one hydrophobic polymer, or combinations thereof.

In one embodiment, the first polymer component includes a first polymer that is a polysaccharide having one or more aldehyde groups. In a certain embodiment, the first polymer component includes a first polymer that is a hydrophilic polymer having one or more aldehyde groups. In another embodiment, the first polymer component includes a first polymer that is a polysaccharide having one or more aldehyde groups, and a hydrophilic polymer. In further embodiments, the first polymer component includes a first polymer that is a polysaccharide having one or more aldehyde groups, a hydrophilic polymer, and a hydrophobic polymer. In some embodiments, the first polymer component comprises a first polymer that includes a polysaccharide and a hydrophilic polymer, wherein both the polysaccharide and hydrophilic polymer have one or more aldehyde groups. Therefore, as used herein, the phrase “first polymer” refers to the one or more polymers of the first polymer component that include one or more functional groups, e.g., aldehydes, that are capable of reacting with a biological tissue and/or the functional groups of the dendrimer component and/or second polymer component. In still further embodiments, the first polymer component comprises a first polymer that includes a polysaccharide and a hydrophilic polymer, wherein both the polysaccharide and hydrophilic polymer have one or more aldehyde groups, and a hydrophobic polymer.

In embodiments, the first polymer comprises at least one polysaccharide. The at least one polysaccharide may be linear, branched, or have both linear and branched sections within its structure. The at least one polysaccharide may be anionic, cationic, nonionic, or a combination thereof. Generally, the at least one polysaccharide may be natural, synthetic, or modified—for example, by crosslinking, altering the polysaccharide's substituents, or both. In one embodiment, the at least one polysaccharide is plant-based. In another embodiment, the at least one polysaccharide is animal-based. In yet another embodiment, the at least one polysaccharide is a combination of plant-based and animal-based polysaccharides. Non-limiting examples of polysaccharides include, but are not limited to, dextran, dextrin, chitin, starch, agar, cellulose, hyaluronic acid, derivatives thereof, such as cellulose derivatives, or a combination thereof.

In embodiments, the at least one polysaccharide is nonionic. Non-limiting examples of nonionic polysaccharides include dextran, dextrin, and cellulose derivatives. In other embodiments, the at least one polysaccharide is anionic. Non-limiting examples of anionic polysaccharides include hyaluronic acid, chondroitin sulfate, alginate, and cellulose gum. In further embodiments, the at least one polysaccharide is cationic. The cationic character may be imparted by substituting the at least one polysaccharide with positively charge groups, such as trimethylammonium groups. Non-limiting examples of cationic polysaccharides include chitosan, cationic guar gum, cationic hydroxyethylcellulose, or other polysaccharides modified with trimethylammonium groups to confer positive charge.

In embodiments, the first polymer component comprises one or more hydrophilic polymers. The hydrophilic polymers are modified, in some embodiments, to confer degradability. For example, the hydrophilic polymers may be modified with polyester groups in order to impart degradability of the hydrophilic polymer. In particular embodiments, the hydrophilic polymers are substituted with one or more functional groups, such as aldehydes, that are capable of reacting with biological tissue and/or the functional groups of the dendrimer and/or second polymer component, such as amines. Generally, any biocompatible hydrophilic polymer may be used. Non-limiting examples of hydrophilic polymers include poly(vinyl alcohol), poly(acrylic acid), poly(acrylamide), poly(ethylene oxide), or combinations thereof.

In embodiments, the first polymer component comprises one or more hydrophobic polymers. The hydrophobic polymers may be modified with pendant hydrophilic polymers to adjust their characteristics. Non-limiting examples of hydrophobic polymers include polycaprolactam, poly(lactic acid), polycaprolactone, or combinations thereof.

In certain embodiments, the first polymer has a molecular weight of about 1,000 to about 1,000,000 Daltons. In one embodiment, the first polymer has a molecular weight of about 5,000 to about 15,000 Daltons. Unless specified otherwise, the “molecular weight” of the polymer refers to the number average molecular weight. The molecular weight may be adjusted to attain certain properties, as known to those of skill in the art.

Generally, the one or more functional groups of the first polymer may be present in a number sufficient to form the hydrogel composites and compositions described herein. In certain embodiments, the first polymer's degree of functionalization is adjustable. The “degree of functionalization” generally refers to the number or percentage of groups on the polymer that are replaced or converted to the desired one or more functional groups. The one or more functional groups, in particular embodiments, include aldehydes. In one embodiment, the degree of functionalization is adjusted based on the type of tissue to which the hydrogel composites or compositions is applied, the concentration(s) of the various components, and/or the type of polymer(s) or dendrimer(s) used in the first and second solutions. In one embodiment, the degree of functionalization is about 10% to about 75%. In another embodiment, the degree of functionalization is about 25% to about 60%. In yet another embodiment, the degree of functionalization is about 40% to about 50%.

In one embodiment, the first polymer is a polysaccharide having about 10% to about 75% of its vicinal hydroxyl groups converted to aldehydes. In another embodiment, the first polymer is a polysaccharide having about 25% to about 75% of its vicinal hydroxyl groups converted to aldehydes.

In one embodiment, the first polymer is dextran with a molecular weight of about 10 kDa. In another embodiment, the first polymer is dextran having about 50% of its vicinal hydroxyl group converted to aldehydes. In a further embodiment, the first polymer is dextran with a molecular weight of about 10 kDa and about 50% of its vicinal hydroxyl groups converted to aldehydes.

In some embodiments, a polysaccharide and/or hydrophilic polymer is oxidized to include a desired percentage of one or more aldehyde functional groups. Generally, this oxidation may be conducted using any known means. For example, suitable oxidizing agents include, but are not limited to, periodates, hypochlorites, ozone, peroxides, hydroperoxides, persulfates, and percarbonates. In one embodiment, the oxidation is performed using sodium periodate. Typically, different amounts of oxidizing agents may be used to alter the degree of functionalization. In addition to, or independently of, other methods, aldehyde groups can be grafted onto the polymer backbone using known bioconjugation techniques in the event that oxidative methods are unsuitable.

### Second Polymer Component

The second polymer component generally includes a second polymer with one or more functional groups capable of reacting with one or more functional groups of the first polymer of the first polymer component. The second polymer component, in embodiments, comprises a second polymer having one or more amines. The amines may be primary amines, secondary amines, or a combination thereof.

The polymers of the second polymer component may be selected from any biocompatible polymers capable of forming or imparting certain characteristics to the hydrogel composites and compositions described herein. The polymers of the second polymer component, for example, may be selected from at least one biopolymer, polyamine, or a combination thereof.

In one embodiment, the second polymer component includes a second polymer that is a biopolymer having one or more amines, such as primary amines, secondary amines, or a combination thereof. Non-limiting examples of biopolymers include chitosan, collagen, gelatin, other structural biomolecules, or a combination thereof. In a particular embodiment, the second polymer comprises a polyamine. The polyamine may be synthetic. Non-limiting examples of polyamines include amine-terminated, multi-arm poly(ethylene oxide) and polyethyleneimine. In another embodiment, the second polymer component includes a second polymer that comprises both (i) a biopolymer having one or more amines, and (ii) a polyamine. Therefore, as used herein, the phrase “second polymer” refers to the one or more polymers of the second polymer component that include one or more functional groups, e.g., amines, that are capable of reacting with the one or more functional groups of the first polymer component, such as aldehydes.

In some embodiments, the second polymer is a commercially available amine-terminated polymer, such as Type I collagen, Type II collagen, Type III collagen, gelatin that is acid- or base-catalyzed (i.e., Type A or Type B), or 10 kD dextran (Pharmacosmos A/S, Denmark).

### Dendrimer Component

In embodiments, the second solution comprises a dendrimer component. The dendrimer component may include a dendrimer that may be substituted with one or more functional groups, such as amines, that are capable of reacting with the one or more functional groups of the first polymer of the first polymer component.

In some embodiments, the dendrimer has amines on at least a portion of its surface groups, which are commonly referred to as “terminal groups” or “end groups.” The dendrimer may have amines on from 25% to 100% of its surface groups. In some embodiments, the dendrimer has amines on 100% of its surface groups. In one embodiment, the dendrimer has amines on less than 75% of its surface groups. As used herein, the term “dendrimer” refers to any compound with a polyvalent core covalently bonded to two or more dendritic branches. In some embodiments, the polyvalent core is covalently bonded to three or more dendritic branches. In one embodiment, the amines are primary amines. In another embodiment, the amines are secondary amines. In yet another embodiment, one or more surface groups have at least one primary and at least one secondary amine.

In one embodiment, the dendrimer extends through at least 2 generations. In another embodiment, the dendrimer extends through at least 3 generations. In yet another embodiment, the dendrimer extends through at least 4 generations. In still another embodiment, the dendrimer extends through at least 5 generations. In a further embodiment, the dendrimer extends through at least 6 generations. In still a further embodiment, the dendrimer extends through at least 7 generations.

In one embodiment, the dendrimer has a molecular weight of about 1,000 to about 1,000,000 Daltons. In a further embodiment, the dendrimer has a molecular weight of about 3,000 to about 120,000 Daltons. In another embodiment, the dendrimer has a molecular weight of about 10,000 to about 100,000 Daltons. In yet another embodiment, the dendrimer has a molecular weight of about 20,000 to about 40,000 Daltons. Unless specified otherwise, the “molecular weight” of the dendrimer refers to the number average molecular weight.

Generally, the dendrimer may be made using any known methods. In one embodiment, the dendrimer is made by oxidizing a starting dendrimer having surface groups comprising at least one hydroxyl group so that at least a portion of the surface groups comprise at least one amine. In another embodiment, the dendrimer is made by oxidizing a starting generation 5 (G5) dendrimer having surface groups comprising at least one hydroxyl group so that at least a portion of the surface groups comprise at least one amine. In yet another embodiment, the dendrimer is made by oxidizing a starting G5 dendrimer having surface groups comprising at least one hydroxyl group so that about 25% to 100% of the surface groups comprise at least one amine. In a particular embodiment, the dendrimer is a G5 dendrimer having primary amines on about 25% to 100% of the dendrimer's surface groups. In a certain embodiment, the dendrimer is a G5 dendrimer having primary amines on about 25% of the dendrimer's surface groups.

In one embodiment, the dendrimer is a poly(amidoamine)-derived (PAMAM) dendrimer. In another embodiment, the dendrimer is a G5 PAMAM-derived dendrimer. In yet another embodiment, the dendrimer is a G5 PAMAM-derived dendrimer having primary amines on about 25% to 100% of the dendrimer's surface groups. In a further embodiment, the dendrimer is a G5 PAMAM-derived dendrimer having primary amines on about 25% of the dendrimer's surface groups.

In one embodiment, the dendrimer is a poly(propyleneimine)-derived dendrimer.

In some instances, at least one of the first solution, the first polymer component, the second solution, the second polymer component, and the dendrimer further includes one or more additives. Generally, the amount of additive may vary depending on the application, tissue type, concentration of the dendrimer in the second solution, the type of dendrimer, concentration of the second polymer component in the second solution, the type of second polymer component, the type of first polymer component, and/or the concentration of the first polymer component in the first solution. Example of suitable additives, include but are not limited to, pH modifiers, thickeners, antimicrobial agents, colorants, surfactants, and radio-opaque compounds. Specific examples of these types of additives are described herein. In one embodiment, at least one of the first solution, the first polymer component, the second solution, the second polymer component, and the dendrimer comprises a foaming additive.

In particular embodiments, at least one of the first solution, the first polymer component, the second solution, the second polymer component, and the dendrimer includes one or more drugs. In such embodiments, the hydrogel composites or compositions may serve as a matrix material for controlled delivery of the one or more drugs. The one or more drugs may be essentially any pharmaceutical agent suitable for local, regional, or systemic administration from a quantity of the hydrogel composite or composition that has been applied to one or more tissue sites in a patient. In one embodiment, the one or more drugs comprises a thrombogenic agent. Non-limiting examples of thrombogenic agents include thrombin, fibrinogen, homocysteine, estramustine, and combinations thereof. In another embodiment, the one or more drugs comprises an anti-inflammatory agent. Non-limiting examples of anti-inflammatory agents include indomethacin, salicyclic acid acetate, ibuprophen, sulindac, piroxicam, naproxen, and combinations thereof.

In other particular embodiments, at least one of the first solution, the first polymer component, the second solution, the second polymer component, and the dendrimer includes one or more cells. For example, in any of these embodiments, the hydrogel composites or compositions may serve as a matrix material for delivering cells to a tissue site at which the hydrogel composites or compositions have been applied. In embodiments, the cells may comprise endothelial cells (EC), endothelial progenitor cells (EPC), hematopoietic stem cells, or other stem cells. In one embodiment, the cells are capable of releasing factors to treat cardiovascular disease and/or to reduce restenosis. Other types of cells are envisioned.

### Formation of Hydrogel Composites and Compositions

Generally, the hydrogel composites and compositions described herein may be formed by combining the first solution and the second solution in any manner. In some embodiments, the first solution, and the second solution are combined before contacting a biological tissue. In other embodiments, the first solution, and the second solution are combined, in any order, on or in a biological tissue. In further embodiments, the first solution is applied to a first biological tissue, the second solution is applied to a second biological tissue, and the first and second biological tissues are contacted. In still a further embodiment, the first solution is applied to a first region of a biological tissue, the second solution is applied to a second region of a biological tissue, and the first and second regions are contacted.

Generally, the hydrogel composites and compositions may be applied to a biological tissue as a drug delivery composition. The hydrogel composites and compositions also may be configured as a tissue adhesive or sealant.

The hydrogel composites and compositions may be applied to the biological tissue using any suitable tool and methods. Non-limiting examples include the use of syringes or spatulas. Double barrel syringes with rigid or flexible discharge tips, and optional extension tubes, known in the art are envisioned.

After the hydrogel composites and compositions have been applied to one or more biological tissues, the first metal nanoparticle may be exposed to one or more wavelengths of light that are (i) converted to heat by the first metal nanoparticle, (ii) effective to release the drug from the first metal nanoparticle, or (iii) a combination thereof. The one or more wavelengths may include one or more infrared wavelengths, or one or more near-infrared wavelengths. In one embodiment, the light has a wavelength of about 800 nm to about 820 nm, about 805 nm to about 815 nm, or about 808 nm.

The first metal nanoparticles may be exposed to the one or more wavelengths at any time after a hydrogel or a portion thereof that includes a first metal nanoparticle is applied to one or more biological tissues. For example, a first metal nanoparticle may be exposed to the one or more wavelengths of light 1 day, 2 days, 3 days, 4 days, 5 days, 6 days, 7 days, 8 days, 9 days, 10 days, or a combination thereof after a hydrogel or a portion thereof has been applied to a biological tissue. When the first metal nanoparticles are exposed to the one or more wavelengths more than once, the duration of each exposure may be effective to [1] release only a portion of the drug conjugated to the first metal nanoparticles, or [2] only generate heat, which may kill and/or damage a diseased cell. Alternatively, the first metal nanoparticles may be exposed to the one or more wavelengths only once, and the duration of the exposure may ensure that substantially all of the drug is released from the first metal nanoparticles. For example, a first metal nanoparticle may be exposed to the one or more wavelengths for about 5 second to about 120 seconds, about 10 to about 120 seconds, about 30 to about 120 seconds, about 30 to about 90 seconds, or about 60 to about 90 seconds. In a particular embodiment, the one or more wavelengths of light includes an 808 nm laser light source (1 W). The first metal nanoparticle may be exposed to one or more wavelengths of light to generate heat, which kill and/or damage a diseased cell, even after substantially all of the drug has been released.

As used herein, the hydrogel composites and compositions are a “treatment” when they improve the response of at least one biological tissue to which they are applied. In some embodiments, the improved response is slowing or reversing tumor growth, eliminating or reducing the likelihood of cancer recurrence, inducing cytotoxicity in cancer cells, lessening overall inflammation, improving the specific response at the wound site/interface of the tissue and hydrogel composites or compositions, enhancing healing, repairing torn or broken tissue, or a combination thereof. As used herein, the phrase “lessening overall inflammation” refers to an improvement of histology scores that reflect the severity of inflammation. As used herein, the phrase “improving the specific response at the wound site/interface of the tissue and hydrogel composite or compositions” refers to an improvement of histology scores that reflect the severity of serosal neutrophils. As used herein, the phrase “enhancing healing” refers to an improvement of histology scores that reflect the severity of serosal fibrosis.

In embodiments, the hydrogel composites and compositions may be used in challenging or awkward implantation environments, including under flowing liquids and/or in inverted geometries.

Before or after contacting one or more biological tissues, the hydrogel composites and compositions may be allowed adequate time to cure or gel. When the hydrogel composites and compositions “cure” or “gel,” as those terms are used herein, it means that the one or more functional groups of the first polymer have undergone one or more reactions with the dendrimer and/or second polymer, and one or more biological tissues. Not wishing to be bound by any particular theory, it is believed that the hydrogel composites and compositions described herein are effective because the first polymer component reacts with both (i) the dendrimer and/or second polymer component, and (ii) the surface of the biological tissues. In certain embodiments, the first polymer component's aldehyde functional groups react with the amines on (i) the dendrimer and/or second polymer component, and (ii) the biological tissues to form imine bonds. In these embodiments, it is believed that the amines on the dendrimer and/or second polymer component react with a high percentage of the aldehydes of the first polymer component, thereby reducing toxicity and increasing biocompatibility of the hydrogel composites and compositions. Typically, the time needed to cure or gel the hydrogel composites and compositions will vary based on a number of factors, including, but not limited to, the characteristics of the first polymer component, second polymer component and/or dendrimer, the concentrations of the first solution and second solution, the pH of the first and second solution, and the characteristics of the one or more biological tissues. In embodiments, the hydrogel composites and compositions will cure sufficiently to provide desired bonding or sealing shortly after the components are combined. The gelation or cure time should provide that a mixture of the components can be delivered in fluid form to a target area before becoming too viscous or solidified and then once applied to the target area sets up rapidly thereafter. In one embodiment, the gelation or cure time is less than 120 seconds. In another embodiment, the gelation or cure time is between 3 and 60 seconds. In a particular embodiment, the gelation or cure time is between 5 and 30 seconds. The hydrogel may be at least partially cured into a shape, such as a disc, prior to being applied to one or more biological tissues.

### Tissue Specific Formulations

Generally, the hydrogel composites and compositions may be adjusted in any manner to compensate for differences between tissues. In one embodiment, the amount of first polymer component is increased or decreased while the amount of dendrimer and/or second polymer component is unchanged. In another embodiment, the amount of dendrimer and/or second polymer component is increased or decreased while the amount of first polymer component is unchanged. In another embodiment, the concentration of the first polymer component in the first solution is increased or decreased while the second solution is unchanged. In yet another embodiment, the concentration of the dendrimer and/or second polymer component in the second solution is increased or decreased while the first solution is unchanged. In a further embodiment, the concentrations of the both the first polymer component in the first solution and the dendrimer and/or second polymer component in the second solution are changed.

When the amine density on the surface of a particular biological tissue is unknown due to disease, injury, or otherwise, an excess of the first solution may, in some embodiments, be added when the hydrogel composites and compositions are first applied, then the amount of first solution may be reduced, e.g., incrementally or drastically, until the desired effect is achieved. The “desired effect,” in this embodiment, may be an appropriate or adequate curing time, adhesion, sealing, treatment, drug delivery, or a combination thereof. Not wishing to be bound by any particular theory, it is believed that an excess of the first solution may be required, in some instances, to obtain the desired effect when the amine density on a biological tissue is low. Therefore, adding an excess will help the user, in this embodiment, achieve adequate sealing or adhesion or treatment in less time.

In other embodiments, however, a lower amount of the first solution may be added when the hydrogel composites and compositions are first applied, then the amount of first solution may be increased, e.g., incrementally or drastically, until the desired effect is achieved, which may be adequate curing time, adhesion, sealing, treatment, or a combination thereof.

In embodiments, the hydrogel composites and compositions can be optimized in view of a target biological tissue, by adjusting one or more of the following: rheology, mechanics, chemistry/adhesion, degradation rate, drug delivery, and bioactivity. These can be adjusted, in embodiments, by altering the type and/or concentration of a first metal nanoparticle and/or a second metal nanoparticle, the type and/or concentration of the first polymer component, and type and/or concentration of the dendrimer, the type and/or concentration of the second polymer component, or a combination thereof.

### Hydrogel Composite and Composition Kits

In another aspect, a kit is provided that comprises a first part that includes the first solution, and a second part that includes the second solution. The kit may further include an applicator or other device means, such as a multi-compartment syringe, for storing, combining, and delivering the two solutions and/or the resulting hydrogel composites and compositions to a tissue site.

In one embodiment, the kit comprises separate reservoirs for the first solution and the second solution. In certain embodiments, the kit comprises reservoirs for first solutions of different concentrations. In other embodiments, the kit comprises reservoirs for second solutions of different concentrations.

In one embodiment, the kit comprises instructions for selecting an appropriate concentration or amount of at least one of the first solution and/or second solution to compensate or account for at least one characteristic of one or more biological tissues. In one embodiment, the hydrogel composites and compositions are selected based on one or more predetermined tissue characteristics. For example, previous tests, may be performed to determine the number of density of bonding groups on a biological tissue in both healthy and diseased states. Alternatively, a rapid tissue test may be performed to assess the number or density of bonding groups. Quantification of tissue bonding groups can be performed by contacting a tissue with one or more materials that (1) have at least one functional group that specifically interacts with the bonding groups, and (2) can be assessed by way of fluorescence or detachment force required to separate the bonding groups and the material. In another embodiment, when the density of bonding groups on a biological tissue is unknown, an excess of the first polymer having one or more aldehydes, may be initially added as described herein to gauge the density of bonding groups on the surface of the biological tissue.

In certain embodiments, the kit comprises at least one syringe. In one embodiment, the syringe comprises separate reservoirs for the first solution and second solution. The syringe may also comprise a mixing tip that combines the two solutions as the plunger is depressed. The mixing tip may be release-ably securable to the syringe (to enable exchange of mixing tips), and the mixing tip may comprise a static mixer. In some embodiments, the reservoirs in the syringe may have different sizes or accommodate different volumes of solution. In other embodiments, the reservoirs in the syringe may be the same size or accommodate the same volumes of the solution.

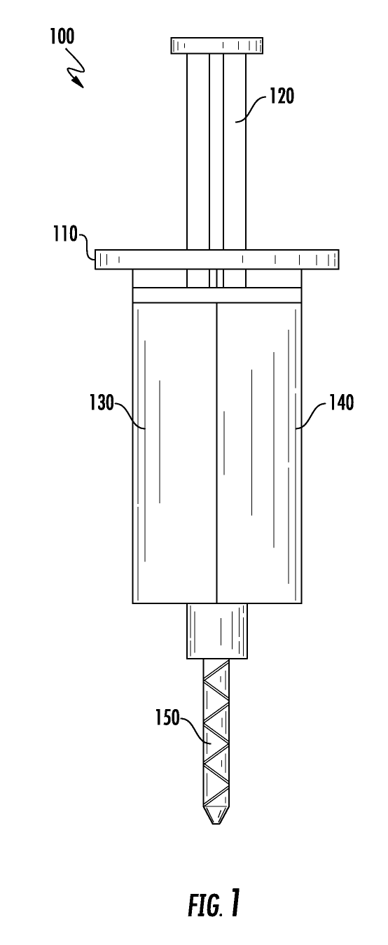

FIG. 1 depicts one embodiment of a syringe 100. The syringe 100 includes a body 110 with two reservoirs (130, 140). A first solution is disposed in the first reservoir 130, and a second solution is disposed in the second reservoir 140. The two reservoirs (130, 140) are emptied by depressing the plunger 120, which pushes the contents of the two reservoirs (130, 140) into the mixing tip 150 and out of the syringe 100.

In a further embodiment, one or more of the reservoirs of the syringe may be removable. In this embodiment, the removable reservoir may be replaced with a reservoir containing a first solution or second solution of a desired concentration.

In a preferred embodiment, the kit is sterile. For example, the components of the kit may be packaged together, for example in a tray, pouch, and/or box. The packaged kit may be sterilized using known techniques at suitable wavelengths (where applicable), such as electron beam irradiation, gamma irradiation, ethylene oxide sterilization, or other suitable techniques.

The kit also may include a light source. The light source may emit one or more wavelengths of light that are capable of being converted to heat by the first metal nanoparticle, cause the release of drug from the first metal nanoparticle, or a combination thereof. The light source may be a NIR laser.

In the descriptions provided herein, the terms “includes,” “is,” “containing,” “having,” and “comprises” are used in an open-ended fashion, and thus should be interpreted to mean “including, but not limited to.” When methods and composite materials are claimed or described in terms of “comprising” various components or steps, the composite materials and methods can also “consist essentially” of or “consist of” the various components or steps, unless stated otherwise.

The terms “a,” “an,” and “the” are intended to include plural alternatives, e.g., at least one. For instance, the disclosure of “a first metal nanoparticle,” “a second metal nanoparticle,” “a targeting biomolecule”, and the like, is meant to encompass one, or mixtures or combinations of more than one first metal nanoparticle, second metal nanoparticle, targeting biomolecule, and the like, unless otherwise specified.

Various numerical ranges may be disclosed herein. When Applicant discloses or claims a range of any type, Applicant's intent is to disclose or claim individually each possible number that such a range could reasonably encompass, including end points of the range as well as any sub-ranges and combinations of sub-ranges encompassed therein, unless otherwise specified. Moreover, all numerical end points of ranges disclosed herein are approximate. As a representative example, Applicant discloses, in one embodiment, that the gold nanosphere has an average diameter of about 25 nm to about 35 nm. This range should be interpreted as encompassing average diameters in a range from about 25 nm to about 35 nm, and further encompasses “about” each of 25 nm, 26 nm, 27 nm, 28 nm, 29 nm, 30 nm, 31 nm, 32 nm, 33 nm, 34 nm, and 35 nm, including any ranges and sub-ranges between any of these values.

### EXAMPLES

The present invention is further illustrated by the following examples, which are not to be construed in any way as imposing limitations upon the scope thereof. On the contrary, it is to be clearly understood that resort may be had to various other aspects, embodiments, modifications, and equivalents thereof which, after reading the description herein, may suggest themselves to one of ordinary skill in the art without departing from the spirit of the present invention or the scope of the appended claims. Thus, other aspects of this invention will be apparent to those skilled in the art from consideration of the specification and practice of the invention disclosed herein.

In the following examples, the general characterization of the nanoconjugates and hydrogel scaffolds was performed by dynamic light scattering (DLS; Wyatt Dyna Pro Plate Reader), zeta potential (Zetasizer Nano-Z S90 Malvern), fluorescence and UV/Vis spectroscopy, environmental scanning microscopy (FEI/Philips XL30 FEG ESEM), and High-resolution Cryo-TEM (JEOL 2100 FEG TEM).

In the following examples, the differences between groups were examined using a two-tailed Student's t-test, two-way analysis of variance (ANOVA) or Log-Rank Mantel-Cox test through SPSS statistical package (version 23, SPSS Inc.). All error bars used in this report are mean±SD of at least 3 independent experiments. Statistically significant P values were indicated in figures and/or legends as ***, P<0.005; **, P<0.01; *, P<0.05. All in vivo experiments used 5 mice per treatment group. A power analysis was performed using G*Power software (version 3.1.9.2) to determine sample size. For n=5 mice per group the power is 90% with a signal-to-noise ratio of 2.4, assuming a 5% significance level and a two-sided test. The signal/noise ratio of 2.4 corresponds to a 6.1% background signal. No randomization or blind events were used in animal studies.

### Example 1

### Functionalization of Gold Nanoparticles with Poly(Ethylene Glycol) (PEG)

10 nM of bare gold nanoparticles (40×10 nm gold nanorods from Nanopartz™ Inc. or 20 nm gold nanospheres from Cytodiagnostics Inc.) dispersed in aqueous solution of 18 MEG DI Water were mixed with 0.006 mg/mL of a commercial hetero-functional PEG (α-Mercapto-ω-carboxy PEG solution, HS—C₂H₄—CONH-PEG-O—C₃H₆—COOH, MW. 3.5 kDa, Sigma) in an aqueous solution of SDS (0.08%). PEG excess was removed by centrifugation (20.000×g, 30 min, 4° C.) and quantified by the Ellman's Assay. The excess of thiolated chains in the supernatant was quantified by interpolating a calibration curve set by reacting 200 μL of α-Mercapto-ω-carboxy PEG solution in 100 μL of phosphate buffer (0.5 M, pH 7) with 7 μL 5,5′-dithio-bis(2-nitrobenzoic) acid (DTNB, 5 mg/mL) in phosphate buffer (0.5 M, pH 7) and measuring the absorbance at 412 nm after 10 minutes reaction.

The linear range for the PEG chain obtained by the method of this example was 0-0.1 mg/mL (Abs at 412 nm=6.9016×[PEG, mg/mL]+0.0536). The number of exchanged chains was given by the difference between the amount determined by this assay and the initial amount incubated with the nanoparticles. There was a point at which the nanoparticle was believed to become saturated with a thiolated layer and was not able to take up more thiolated chains—maximum coverage per gold nanoparticle, i.e. 0.03 mg/mL of PEG for both rods and spheres.

The gold nanorods were functionalized with a 100% saturated PEG layer as all the biomolecules attached were functionalized via chemical modification in the PEG layer. The gold nanospheres were functionalized with a 50% PEG layer to allow for space to bind the thiolated siRNAs.

### Example 2

### Functionalization of Gold Nanospheres with siRNA Molecules

The gold nanospheres of Example 1 were functionalized with siRNAs, and used in conjunction with the nanorods to deliver the siRNAs. An siRNA was selected that silences a major oncogene driver in CRC progression: Kras (Kirsten Rat Sarcoma Viral Oncogene Homolog).

Kras is an effector molecule responsible for signal transduction in colorectal cancer and approximately 30% to 50% of colorectal tumors are known to have a mutated Kras gene (see, e.g., Sunakawa, Y., et al. New England Journal of Medicine 369, 2159-2159 (2013)).

The gold nanospheres of Example 1 were functionalized with siRNA labelled with DY647 against Kras gene. Thiolated siRNA (Thermo Scientific Dharmacon) was dissolved in 1 ml of 0.1 M DTT, extracted three times with ethyl acetate, and further purified through a desalting NAP-5 column (Pharmacia Biotech) according to the manufacturer's instructions. The siRNA was only resuspended in DEPC-water and incubated immediately with gold nanospheres previously functionalized with PEGs. The purified thiolated siRNAs were incubated at a concentration of 10 μM with an RNase-free solution of the PEG-gold nanospheres (10 nM) of Example 1 containing 0.08% SDS. Subsequently, the salt concentration was increased from 0.05 to 0.3 M NaCl with brief ultrasonication following each addition, which increased the coverage of oligonucleotides on the nanoparticle surface. After functionalization during 16 hours at 4° C., the particles were purified by centrifugation (20.000×g, 20 mins, 4° C.), and re-suspended in DEPC-water. This procedure was repeated 3 times.

The number of siRNA per nanoparticle was determined by quantification of the excess siRNA oligos in the supernatants collected during synthesis via the emission spectra of DY647 (excitation/emission, 645 nm/663 nm) dye in a microplate reader (Varioskan Flash Multimode Reader; Thermo Scientific). All nanoparticle samples and standard solutions of the thiolated-siRNAs were kept at the same pH and ionic strength for all measurements. Fluorescence emission was converted to molar concentrations by interpolation from a standard linear calibration curve prepared with known concentrations of siRNA.

### Example 3

### Functionalization of Gold Nanorods with Avastin®

Gold nanorods, which had the capacity to convert near-infrared (NIR) radiation into heat and thereby cause localized release, were functionalized with the Food and Drug Administration (FDA) approved monoclonal antibody, Bevacizumab or Avastin®, that blocks vascular endothelial growth factor (VEGF) pathway in CRC.

The antibody Avastin® (Bevacizumab from Roche) was coupled to the PEG-gold nanorods of Example 1 by a carbodiimide chemistry, assisted by N-hydroxysuccinimide (EDC/NHS coupling reaction), between the carboxylated PEG terminal and the primary amine groups of the antibody.

10 nM of PEG-gold nanorods, 1.98 mg/mL N-hydroxysulfosuccinimide (sulfo-NHS, Sigma) and 500 μg/mL EDC (1-Ethyl-3-(3-dimethylaminopropyl)carbodiimide, Sigma) were incubated in 10 mM MES (2-(N-morpholino)ethanesulfonic acid, Sigma) at pH 6.2 and allowed to react for 30 minutes to activate the carboxylic groups on PEG molecules. Then, activated PEG-gold nanorods were washed once with 10 mM IVIES (pH 6.2) and used immediately. Avastin® was added to the mixture at a final concentration of 15 μg/mL and allowed to react for 16 hours at 25° C. The Avastin®-PEG-gold nanorods were centrifuged at 20.000×g for 30 min at 4° C., and washed three times with Mili-Q water.

The Avastin® coupling was quantified using the Bradford Assay kit (Thermo Scientific) according to the manufacturer's instructions. The Bradford assay was based on the observation that the absorbance maximum for an acidic solution of Coomassie Brilliant Blue G-250 shifts from 465 nm to 595 nm when binding to protein occurs. Both hydrophobic and ionic interactions stabilized the anionic form of the dye, which caused a visible colour change. The standard curve was used to determine the Avastin® concentration of each unknown sample (supernatant).

Bevacizumab (Avastin®) was selected for this embodiment, because it neutralizes VEGF and blocks its signal transduction through VEGFR-1 and VEGFR-2 receptors, as demonstrated by the inhibition of VEGF-induced cell proliferation, survival, permeability, nitric oxide production, migration and tissue factor production (see, e.g., Wang, Y., et al. Angiogenesis 7, 335-345 (2004)). Inhibiting VEGFR activity permitted the blocking of the endogenous expression of VEGF, and a reduction in the amount of secreted VEGF²⁰. The particles were configured to release the drug (Avastin®) and produce heat upon laser irradiation, which may kill any remaining cancer cells.

### Example 4

### HA1 and TCP-1 Peptides Functionalization

TCP-1 peptide (N-CTPSPFSHC-C) is commonly used to target the vasculature of orthotropic CRC patients and specifically targets colorectal cancer cells (see, e.g., Li, Z. J. et al. Journal of Controlled Release 148, 292-302 (2010)). Due at least in part to these features, TCP-1 peptide was conjugated to the nanorods and the nanospheres in an effort to provide preferential uptake in cancer cells.

The TCP-1 (N-CTPSPFSHC-C) and HA1 (N-YPYDVPDYA-C) peptides were functionalized to the gold nanoparticles of Example 1 by a carbodiimide chemistry assisted by N-hydroxisuccinimide using an EDC/NHS coupling reaction between the carboxylated PEG spacer and the amine terminal group of the peptides.

The TCP-1 peptide, which was specific to target colorectal cancer cells, was conjugated to both the nanorods and nanospheres of Example 1, because doing so likely increased selective binding and uptake to cancer cells.

In an effort to enhance the siRNA uptake, the gold nanospheres were functionalized with an HA1 peptide (in addition to the TCP-1). The HA1 peptide is a fusogenic peptide (Influenza Hemagglutinin—HA1 peptide, N-YPYDVPDYA-C) which destabilizes the endosomal membrane and stimulates endosomal discharge by a pH-responsive mechanism (see, e.g., Bosch, F. X., et al. Virology 113, 725-735 (1981)).

HA1 peptide, which enhanced the siRNA uptake, was functionalized only on the nanospheres of Example 1.

10 nM of NPs-PEG, 1.98 mg/mL N-hydroxysulfosuccinimide (sulfo-NHS, Sigma) and 500 μg/mL EDC (1-Ethyl-3-(3-dimethylaminopropyl)carbodiimide, Sigma) were incubated in 10 mM MES (2-(N-morpholino)ethanesulfonic acid, Sigma) at pH 6.2, and allowed to react for 30 minutes to activate the carboxylic groups. The activated nanoparticles were washed once with 10 mM MES, pH 6.2 and used immediately. TCP-1, or TCP-1 and HA1 was/were added to the mixture at a final concentration of 3 μg/mL and allowed to react for 16 hours at 25° C. After this period, the nanoparticles were centrifuged at 20.000×g for 30 min at 4° C. and washed three times with Mili-Q water.

The TCP-1 and HA1 quantification was achieved using the Pierce BCA Protein Assay kit (Thermo Scientific) according to manufacturer's instructions. 0.025 mL of each standard and unknown sample (the supernatants) was mixed with 0.2 mL of the BCA™ Working Reagent (50:1, BCA reagent A:BCA reagent B). The reaction mixture was incubated at 60° C. for 30 minutes. After this period the tubes were cooled down to room temperature and the absorbance measured at 562 nm. The standard curve was used to determine the TCP-1 and HA1 concentration of each unknown sample (supernatant). The calibration curve for a working range (0-125 μg/mL) was given by the following equation Abs562 nm=0.0033×[TCP-1 peptide, μg/mL]+0.3903, R²=0.9969 for TCP-1 peptide and Abs562 nm=0.0037×[HA1 peptide, μg/mL]+0.2662, R²=0.9919 for HA1 peptide.

### Example 5

### Stability of Avastin®-Gold Nanorods

Several stability assays were performed to determine a favorable concentration of Avastin® conjugated to the surface of the PEGylated gold nanorods. A fixed amount of PEG-gold nanorods (10 nM) was incubated with increasing amounts of Avastin® (from 0 to 15 μg/mL). The absorbance spectra showed a red shift of the SPR (surface plasmon resonance) peak, and a decrease in the absorbance in the SPR peak with increasing amounts of Avastin®. The absorbance stabilized from 10 to 15 μg/mL.

This demonstrated a strong association of Avastin® to the PEG-rods, which also was observed by the retardation effect of the functionalized rods with increasing amounts of Avastin® on an electrophoresis gel. The ratio between the small SPR peak of the rods at 515 nm and the large and predominant SPR peak at 805 nm after functionalization with increasing amounts of Avastin® demonstrates that a maximum stability was achieved upon functionalization with 10 and 15 μg/mL, which appeared to validate the results for the SPR shift.

### Example 6

### Characterization of Avastin®-Gold Nanorods

The layer of functionalization of the gold nanorods was easily seen with high resolution electron microscopy. The images depicted the difference between the gold nanorods functionalized with only PEG, and those also functionalized with the targeting peptide and the drug.

TEM images showed that the organic layer (stained with 2% phosphotungstic acid) around the gold nanorods increased dramatically with the peptide and the drug functionalization process. This observation was validated with the SPR shift in the gold nanorods absorbance spectra. A red shift of almost 40 nm was observed in the SPR peak when the PEGylated nanorods were functionalized the targeting peptide and the drug.

Several physical and chemical properties of the functionalized gold nanospheres and gold nanorods of Examples 1-6 are provided at Table 1:

TABLE 1
Physical-chemical properties of functionalized gold nanospheres and gold nanorods

| | | Zeta- | siRNA | TCP-1 | HA1 | Avastin |
|---|---|---|---|---|---|---|
| | Size | Potential | mol per | mol per | mol per | mol per |
| Formulation | (nm)^{a} | (mV)^{b} | particle | particle | particle | particle |
| Rods-PEG | 30.2 ± 1.1 | −28.7 ± 4.5 | NA | 28.3 ± 2.1 | NA | NA |
| Rods-PEG-Avastin | 35.6 ± 2.1 | −23.1 ± 3.8 | NA | 28.3 ± 2.1 | NA | 15.2 ± 1.4 |
| Spheres-PEG | 28.9 ± 3.5 | −20.2 ± 1.5 | NA | 29.5 ± 1.8 | 30.1 ± 1.7 | NA |
| Spheres-PEG- | 32.7 ± 2.4 | −28.6 ± 4.7 | 104.8 ± 2.7 | 29.5 ± 1.8 | 30.1 ± 1.7 | NA |
| siRNA control | | | | | | |
| Spheres-PEG- | 32.7 ± 1.9 | −29.4 ± 5.2 | 105.2 ± 1.3 | 29.5 ± 1.8 | 30.1 ± 1.7 | NA |
| siRNA Kras | | | | | | |

NOTE:
All rods were functionalized with TCP-1 peptide and all the spheres with both TCP-1 and HA1 peptides.
^{a}Determined by Dynamic Light Scattering (DLS).
^{b}Nanoparticles analysed at a concentration of 2 nM in water in a total volume of 1 mL, containing 0.1M KCl.

As shown at Table 1, the siRNA:NP ratio for the gold nanospheres was 105:1 and the Avastin® (drug):NP for the gold nanorods was 15:1. The mean particle diameter of siRNA gold nanospheres was 32.7 (±1.9) nm, and 35.6 (±2.1) nm for the drug gold nanorods as measured by dynamic light scattering (DLS). Both siRNA gold nanospheres −29.4 (±5.2) mV and drug gold nanorods −23.1 (±3.8) mV are anionic as measured by Zeta-potential.

### Example 7

### Quantification of Avastin® Release

To evaluate the thermal effects of both gold nanorods and gold nanospheres using a NIR laser light source (808 nm, 1W) and their potential to serve as photothermal probes in vivo, the temperature of gold nanorods and gold nanospheres in aqueous solution was evaluated over time.

It is well known that gold nanorods having dimensions of 40×10 nm present an absorbance peak in the NIR (around 800 nm), whereas gold nanospheres have a diameter of 20 nm have a surface plasmon resonance (SPR) peak around 530 nm. Therefore, when irradiated in the NIR range, only the gold nanorods would theoretically transduce electromagnetic radiation into heat.

A gold nanorods suspension (10 nM) irradiated for 120 seconds (808 nm, 1W) resulted in 50° C. rise in temperature, with no variation in temperature in the gold nanospheres solution following the same irradiation power. These results demonstrated that the synthesized gold nanorods experience substantial thermal effects at low laser energy irradiation.

To corroborate the triggered conjugated drug release following heat-up of gold nanorods in cells, drug release was quantified as a function of increasing laser exposure time. For this purpose, a 808 nm laser light source (1 W) was applied to the drug gold-nanorods solution for 10, 30, 60, 90 and 120 seconds. The gold nanorods then were centrifuged and the supernatant collected for quantification of the drug released to the solution.

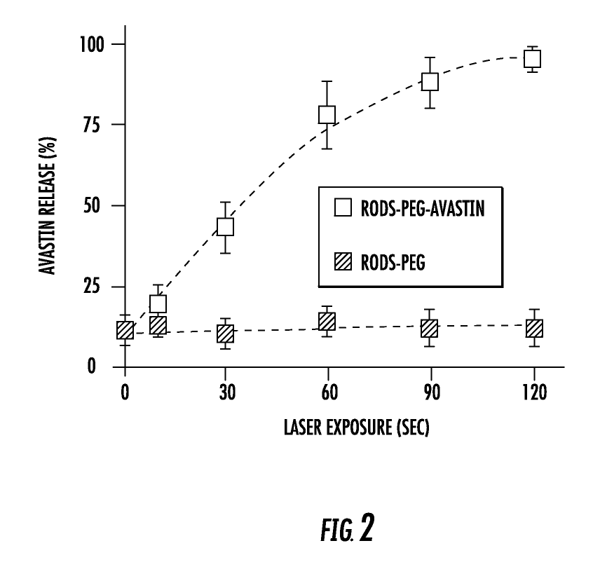

Nearly 95% of the Avastin® was released from the gold nanorods after 120 seconds of laser exposure (FIG. 2).

An analysis of the Avastin® release mechanism revealed that, following NIR laser application, the drug was being released while remaining conjugated to the PEG. This was determined by quantifying the release percentages of thiol-PEG and Avastin® after increasing laser exposure times with the Ellman's assay (for the thiol-PEG) and the Bradford assay (for the) Avastin®), before and after filtration with a centrifugal filter system (Amicon® Ultra-15 30K). This system had a molecular weight cut-off of 30 kDa. The PEG had a number average molecule weight of 3.5 kDa and the Avastin® had a molecular weight of 149 kDa.

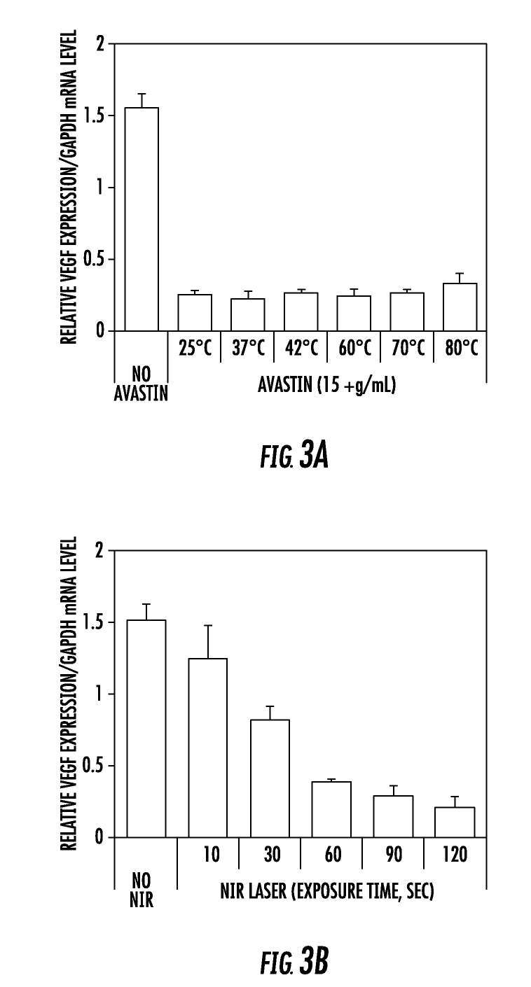

Yet, as shown at FIG. 3A and FIG. 3B, the Avastin® activity in Lovo-6-Luc human colorectal cancer cells was not affected by [1] the exposure to increasing temperatures following laser irradiation (for a range of exposure times), and [2] the drug attachment to the PEG. FIG. 3A depicts the VEGF expression in colorectal cancer cells following treatment with Avastin® antibody previously incubated with increasing temperatures for 2 minutes (the same time as the NIR irradiation time that produced near 100% Avastin release from the nanorods). FIG. 3B depicts the VEGF expression in colorectal cancer cells following treatment with Avastin® released from nanorods previously irradiated for increasing exposure times to a NIR laser. The VEGF mRNA levels were measured by Real-Time PCR using Taqman probes FAM-MGB (Applied Biosystems) and GAPDH as the reference gene. All experiments were done in triplicate and errors reported as standard deviation (s.d.).

The Avastin® (antibody) concentration was evaluated using the BCA Protein Assay (Thermo Scientific) according to the manufacturer's instructions. This assay is a detergent-compatible formulation based on bicinchoninic acid (BCA) for the colorimetric detection and quantification of total protein. This method combines the well-known reduction of Cu⁺² to Cu⁺¹ by protein in an alkaline medium (the biuret reaction) with the highly sensitive and selective colorimetric detection of the cuprous cation (Cu⁺¹) using a unique reagent containing bicinchoninic acid.

The purple-colored reaction product of this assay was formed by the chelation of two molecules of BCA with one cuprous ion. This water-soluble complex exhibited a strong absorbance at 562 nm in a near linear relationship with increasing protein concentrations.

Specifically, 0.1 mL of each standard and unknown sample (the supernatants) was mixed with 2.0 mL of the BCA™ Working Reagent. The reaction mixture was incubated at 60° C. for 2 hours. The tubes were then cooled down to room temperature, and the absorbance was measured at 562 nm. The standard curve was used to determine the Avastin® concentration of each unknown sample (supernatant). The calibration curve for a working range (0-18 μg/ml) was given by the following equation: [Absorbance at 562 nm]=0.0004×[Avastin® Conc., μg/mL]+0.1205; R²=0.9927.

### Example 8

### Cellular Uptake and Efficacy of Nanoparticles

To evaluate cellular uptake, confocal microscopy was performed in Lovo-6-Luc colorectal cancer cells treated with the combination of DY647 labelled siRNA-nanospheres and Alexa-Fluor® 555 labelled drug gold nanorods. Both the siRNA-nanospheres and the drug-nanorods had high cellular uptake efficiency at 24 hours post-incubation.

LoVo-6-Luc-1 colorectal cancer cells (PerkinElmer, tested for mycoplasma contamination by the Division of Comparative Medicine Diagnostic Lab at MIT via IMPACT PCR and was negative) were grown in Ham's F12 medium (Invitrogen) supplemented with 4 mM glutamine, 10% heat inactivated fetal bovine serum (Gibco, Life Technologies), 100 U/ml penicillin and 100 μg/ml streptomycin (Invitrogen) and maintained at 37° C. in 5% CO₂. Cells were seeded at a density of 1×10⁵ cells/well in 24-well plates and grown for 24 hours prior to incubation of gold nanorods and nanospheres (10 nM each). On the day of incubation, the cells were approximately 50% confluent. For confocal microscopy, cells were fixed with 4% paraformaldehyde in PBS for 15 min at 37° C. and stained with DAPI to allow nuclear staining and finally mounted in ProLong® Diamond Antifade Reagent (Invitrogen). Images of cells were taken with a Nikon A1R Ultra-Fast Spectral Scanning Confocal Microscope.

The LoVo-6-Luc-1 colorectal cancer cells incubated with 10 nM of drug-gold nanorods and siRNA-gold nanospheres were washed with PBS and detached with 0.25% trypsin-EDTA (Life Technologies). FACS running buffer (500 μL), consisting of 98% PBS and 2% heat inactivated fetal bovine serum (Gibco, Life Technologies), was added to each well. Cells were mixed thoroughly and then transferred to FACS tube with filter lid, and the Alexa-Fluor® 555 (from gold nanorods) and DY647 (from gold nanospheres) signals were acquired on FACS LSR Fortessa HTS-1 (BD Biosciences) flow cytometer.

Confocal imaging showed strong stability of both nanospheres and nanorods and efficient entry into the target colorectal cancer cells, when compared to the low cellular uptake in healthy 3T3 human fibroblasts. These results demonstrated that both nanoparticles exhibited enhanced cellular uptake in cancer cells when conjugated to the TCP-1 peptide, when compared to healthy cells (see, e.g., Li, Z. J. et al. Journal of Controlled Release 148, 292-302 (2010)).

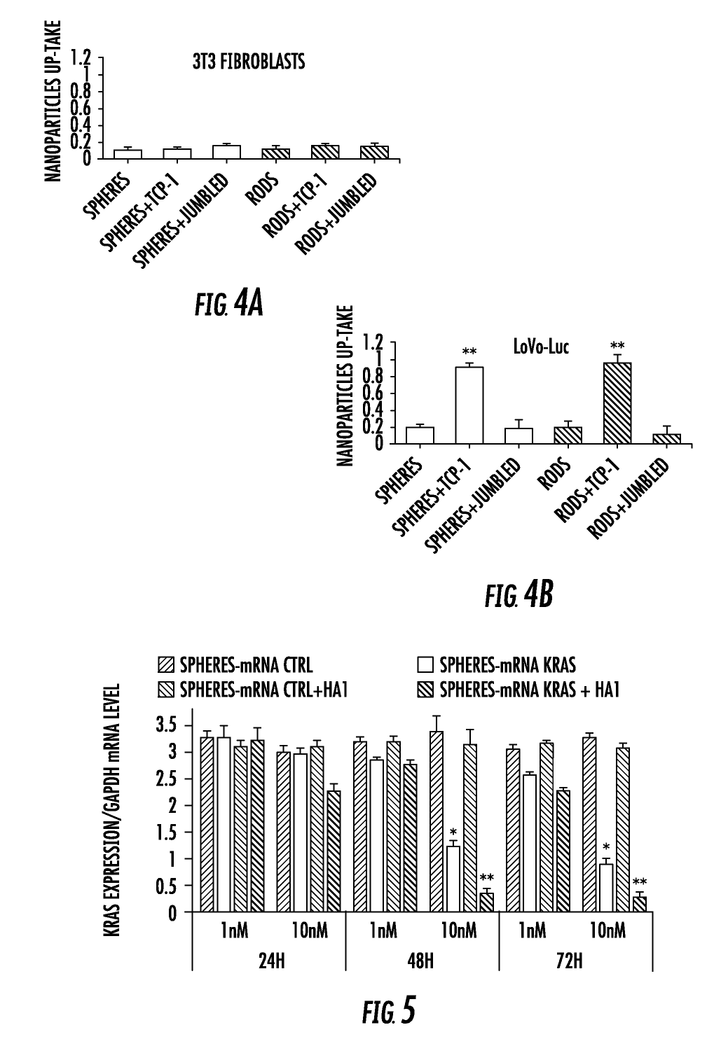

FIG. 4A and FIG. 4B depict the quantification of fluorescence from punctuated structures using Image-J software (version 1.49) of 3T3 fibroblasts (FIG. 4A) and LoVo-Luc colorectal cancer cells (FIG. 4B). All experiments were done in triplicate and error bars plotted as standard deviation (s.d) (n=3, statistical analysis performed with a two-tailed Student's t-test, **, P<0.01).

The effect of siRNA-nanospheres on cancer cells' biochemical activity also was tested. At 24, 48 and 72 h post-transfection, qPCR data confirmed a reduction in Kras expression with increasing amounts of siRNA-nanospheres, as shown at FIG. 5. Specifically, FIG. 5 depicts Kras expression in LoVo-Luc colorectal cancer cells at 24, 48 and 72 hours of incubation with 1, 5 and 10 nM of siRNA gold nanospheres functionalized with and without the HA1 peptide (n=3, statistical analysis was performed with a two-tailed Student's t-test, **, P<0.01; *, P<0.05). Kras levels were normalized to the GAPDH reference gene.

qPCR amplification of mRNA extracted from colorectal cancer cell (Lovo-6-Luc human colorectal adenocarcinoma cells) treated with siRNA-nanospheres confirmed the efficient RNA interference (RNAi) mechanism, as indicated by the decrease in expression of the target genes upon treatment with siRNA-nanospheres. siRNA-nanospheres functionalized with the fusogenic peptide HA1 were more efficient in downregulating Kras, confirming the peptide function in enhancing nanospheres' uptake and hence siRNA availability in the cytoplasm.

A live-dead assay of colorectal cancer cells following uptake of 10 nM of spheres, rods, or the combination of spheres and rods 24 h post laser application or following increased exposure time to both NPs was performed. This assay was conducted using a double staining procedure with acridine orange (AO) and propidium iodide (PI) representing green and red fluorescence for live and dead cells, respectively. The treated cells were incubated for 30 minutes with acridine orange (Sigma) that stains live cells at a final concentration of 0.67 μM and with propidium iodide (Sigma) that stains apoptotic/dead cells at a final concentration of 75 μM. Then cells were washed 2× with PBS 1× and images of cells were taken either with a Nikon A1R Ultra-Fast Spectral Scanning Confocal microscope or with a Nikon Eclipse Ti Epi-fluorescence microscope.

The assay revealed that only drug-gold nanorods or the combination of rods and spheres resulted in a vast cell death (>95%) 24 hours after laser application. This likely was due to the combination of local heat and consequent drug (Avastin®) release.

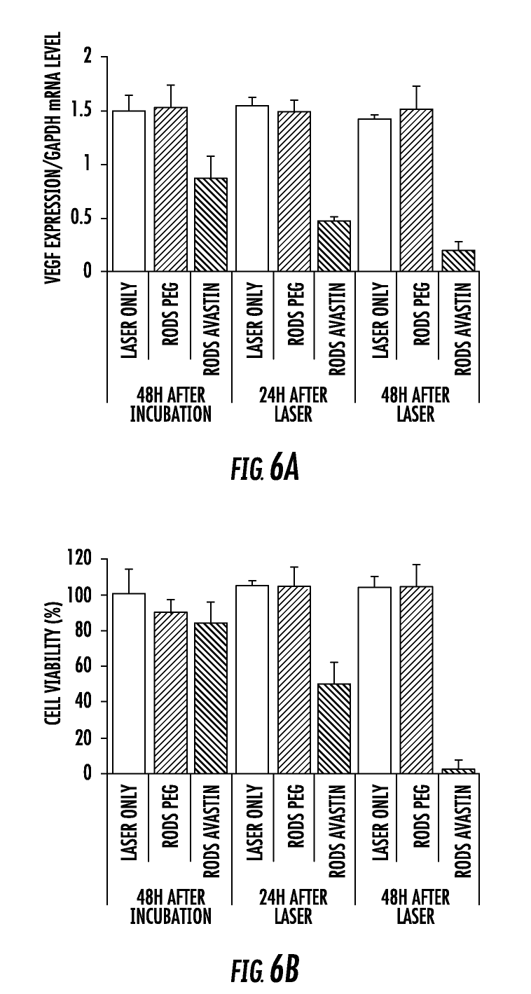

To validate Avastin® release from the gold nanorods and evaluate the efficacy of the drug in blocking VEGF, qPCR was performed with mRNA extracted from cells in the same conditions as described herein for the viability assays. As shown at FIG. 6A, only the cells that were treated with gold nanorods carrying Avastin® were able to inhibit VEGF expression 48 hours after the application of an 808 nm laser light source for 120 seconds. FIG. 6A depicts VEGF expression in colorectal cancer cells following treatment with gold nanorods with and without drug and before and 24 and 48 hours after application of an 808 nm laser light source during 120 seconds. VEGF mRNA levels were measured by Real-Time PCR using TaqMan® probes FAM-MGB (Applied Biosystems) and GAPDH as the reference gene.

The tumorigenicity of the colorectal cancer cells was evaluated by measuring the IC₅₀ via an MTT assay following treatment with gold nanorods with and without drug and prior to or at 24 and 48 hours post-laser irradiation.

A standard MTT [3-(4,5-dimethylthiazol-2-yl)-2,5-diphenyltetrazoliumbromide] reduction assay (Molecular Probes®, Life Technologies) was performed to determine the viability of cells to increasing concentrations of drug-gold nanorods and siRNA-gold nanospheres. Cells were seeded at a density of 1×10⁵ cells per well in 24-well culture plates in complete Ham's F12 medium (500 μl) with serum. After 24 hours of exposure to gold nanorods and nanospheres, the medium was removed and the cells were washed twice with sterile PBS and 300 μL of fresh medium with serum was added. Then 16.7 μl of sterile MTT stock solution (5 mg/mL in PBS) was added to each well. After incubation for an additional 2 hours, the medium was removed and the formazan crystals were resuspended in 300 μl of dimethyl sulfoxide (Sigma). The solution was mixed and its absorbance was measured at 540 nm as a working wavelength and 630 nm as reference using a microplate reader (Varioskan Flash Multimode Reader, Thermo Scientific). The cell viability was normalized to that of cells cultured in the culture medium with PBS treatment.

As shown at FIG. 6B, gold nanorods with Avastin® significantly inhibited the growth of colorectal cancer cells when compared to the cells that were only laser irradiated or cells that were incubated with gold nanorods without Avastin®. FIG. 6B depicts cell viability via an MTT assay following treatment with gold nanorods with and without drug and before and 24 and 48 hours after application of an 808 nm laser light source during 120 seconds. All experiments were done in triplicate and errors reported as standard deviation (s.d.).

### Example 9

### Hydrogel Compositions

Implantable dendrimer-dextran hydrogels were prepared, and the hydrogels were doped with the drug-gold nanorods and siRNA-gold nanospheres of Example 4 to afford local drug/gene delivery and hyperthermia.

To facilitate local release to diseased tissue while avoiding unwanted material migration and release of a substantial amount of drug to one or more adjacent sites, the hydrogel was decorated with aldehyde groups (provided by an oxidized dextran) that interacted with tissue amines to form adhesive bonds.

Equal parts of dendrimer amine of 12.5% solid content and dextran aldehyde 5% solid content with 0.25% fluorescently labelled dextran were mixed to form 6 mm pre-cured disks. For doped scaffolds, 10 nM of drug-gold nanorods and 10 nM siRNA-gold nanospheres were added to the dendrimer solution prior to hydrogel formation. All solutions were filtered through a 0.22 μm filter prior to hydrogel formation for in vivo implantation. Pre-cured disks of fluorescently labelled scaffold with or without RNA nanoparticles were formed and implanted subcutaneously on top of the mammary tumor in mice. For systemic administration 10 nM of drug-gold nanorods and 10 nM siRNA-gold nanospheres was injected in mice via tail-vein and for intratumoral administration 10 nM of drug-gold nanorods and 10 nM siRNA-gold nanospheres was injected directly to the tumor.

Dextran aldehyde (Mr 10,000 Da, 50% oxidation; 10 mg) was tagged by reaction with 2 mg of Alexa-Fluor® 405 Cadaverine (Invitrogen) in 20 mL of 50 mM carbonate buffer (pH 8.5) for 1 hour at room temperature. Then, the reaction crude was cooled down in an ice-water bath and imine bonds were reduced with 20 mL of 30 mM sodium cyanoborohydrate in PBS for 4 hours. Then, tagged dextran aldehyde was dialyzed four times through a 3,000 Da MWCO Centrifugal Filter (Millipore) for 20 min each time at room temperature and 4,000 RCFs. The purified product was lyophilized.

Pre-cured fluorescently labelled scaffolds alone (control) or doped with drug-gold nanorods and siRNA-gold nanospheres were snap-frozen in liquid nitrogen and kept at −80° C. for 24 hours. Then, 12 μm-thick cryosections (Cryostat Leica CM1850) were analysed by fluorescence microscopy (NIS-Elements Nikon). Controls for this experiment included empty scaffold (without nanoparticles).

The adhesive architecture was evaluated by high-resolution scanning electron microscopy (SEM). In addition to the SEM images, an energy-dispersive X-ray spectroscopy (EDS) map-scanning analysis was collected of the hydrogel doped with both drug-gold nanorods and siRNA-gold nanospheres to evaluate their elemental composition, especially the gold content, which pointed at a uniform particle distribution throughout the hydrogel.

### Example 10

### Platform Efficacy

Platform efficacy was examined in an in vivo mouse model of colorectal cancer.

Sub-cutaneous tumors were induced in male SCID hairless congenic mice (SHC™ Mouse CB17.Cg-Prkdc^{scid}Hr^{hr}/IcrCrl from Charles River Laboratories International, 6 weeks, n=5) by injection of 5×10⁶ LoVo-6-Luc-1 colorectal cancer cells stably expressing firefly luciferase, suspended in 50 μL of HBBS (Lonza) solution. For determination of tumor growth, individual tumors were measured using calliper and tumor volume was calculated by: Tumor volume (mm³)=width×(length²)/2. Treatments began when tumor volume reached about 100 mm³.

A tunable hydrogel patch impregnated with drug- and siRNA-nanoparticle conjugates (drug-gold nanorods and siRNA-gold nanospheres) for local gene and drug release in colorectal tumor cells was designed. The efficacy of the triple-therapy based therapeutic platform was compared to gene-, photo- or chemo-therapy alone and with or without the application of NIR laser source. All therapeutic combinations, single, double or triple therapy, were performed with the same concentration of siRNA or drug and at the same therapeutic regimen.

The hydrogel scaffolds of Example 9 were loaded with the specific therapeutic nanoconjugates, and then implanted adjacent to the colorectal tumor of SCID hairless congenic mice (CB17.Cg-Prkdc^{scid}Hr^{hr}/IcrCrl) when the tumors reached a volume of ˜100 mm³.

In a combinatory wave of treatments, an important gene in cancer progression was knocked down. In a second wave of treatment, a potent drug to stop cancer cell proliferation was delivered upon triggered drug release with NIR application, with concomitant tumor ablation using local phototherapy. The hydrogel was implanted 15 days after tumor induction, and NIR treatment was performed at 18, 19, 20, and 25 days after treatment.

To form a hydrogel, a 12.5% PAMAM G5 dendrimer (25% of its surface groups including amines) solution was mixed with an NP-containing 5% dextran aldehyde solution (drug-gold nanorods and/or siRNA-gold nanospheres were at a final concentration of 10 nM each). Drug-gold nanorods and siRNA-gold nanospheres scaffolds showed complete release within 24 hours under physiological conditions in vitro (pH 7.4 and 37° C.). The therapeutic hydrogels loaded with specific nanoconjugates for each therapy were monitored by fluorescence and thermographic imaging.

Having observed the hydrogels' photothermal efficiency, their capability to remain active as powerful optical nanoantennas for photothermal tumor heating 72 hours post NIR application was subsequently investigated. A hydrogel (dendrimer and dextran only) and hydrogels doped with drug-gold nanorods or siRNA-gold nanospheres or a combination thereof (rods and spheres) were implanted into mice-bearing colorectal tumor. Temperatures above 60° C. occurred only in irradiated tumors containing hydrogels doped with drug-gold nanorods or rods and spheres together, whereas the hydrogel (dendrimer and dextran only) or hydrogel doped with siRNA-gold nanospheres displayed maximum surface temperatures of about 33 ° C.

The capability of the triple-therapy to shrink the tumor prior to its resection or prevent tumor recurrence following resection was evaluated in two separate mouse groups; a hydrogel containing both drug-gold nanorods and siRNA-gold nanospheres was implanted [1] on top of the tumor prior to its resection or [2] at the tumor site following resection as a wash-out procedure.

Inhibition of tumor progression was measured by luciferase expression (see Conde, J., et al. Nat Mater 15, 353-363 (2016)), while nanoconjugates' release (drug gold nanorods in green and siRNA gold nanospheres in red) was tracked fluorescently via a live imaging system for 15 days post-hydrogel implantation and 10 days after the first NIR application.

Specifically, non-invasive longitudinal monitoring of tumor progression was followed by scanning mice with the IVIS Spectrum-bioluminescent and fluorescent imaging system (Xenogen XPM-2 Corporation) from mice bearing colorectal tumors (n=5 animals per treated group). 15 minutes before imaging, mice were intraperitoneally injected with 150 μL of D-luciferin (30 mg/mL, Perkin Elmer) in DPBS (Lonza). Whole-animal imaging was performed at the indicated time points—1, 5, 8 and 15 days post hydrogel disks implantation, and at 1, 3 and 10 post NIR application (120 seconds exposure during 4 sessions). Nanoconjugates uptake and biodistribution were examined by quantifying fluorescence images of live mice for 15 days post-implantation and for excised organs (liver, kidneys, spleen, heart, lungs and intestines). No fluorescent signal was detected in any of the major organs. All nanoconjugates accumulated exclusively in the tumor tissue based on the ex vivo images of the organs.

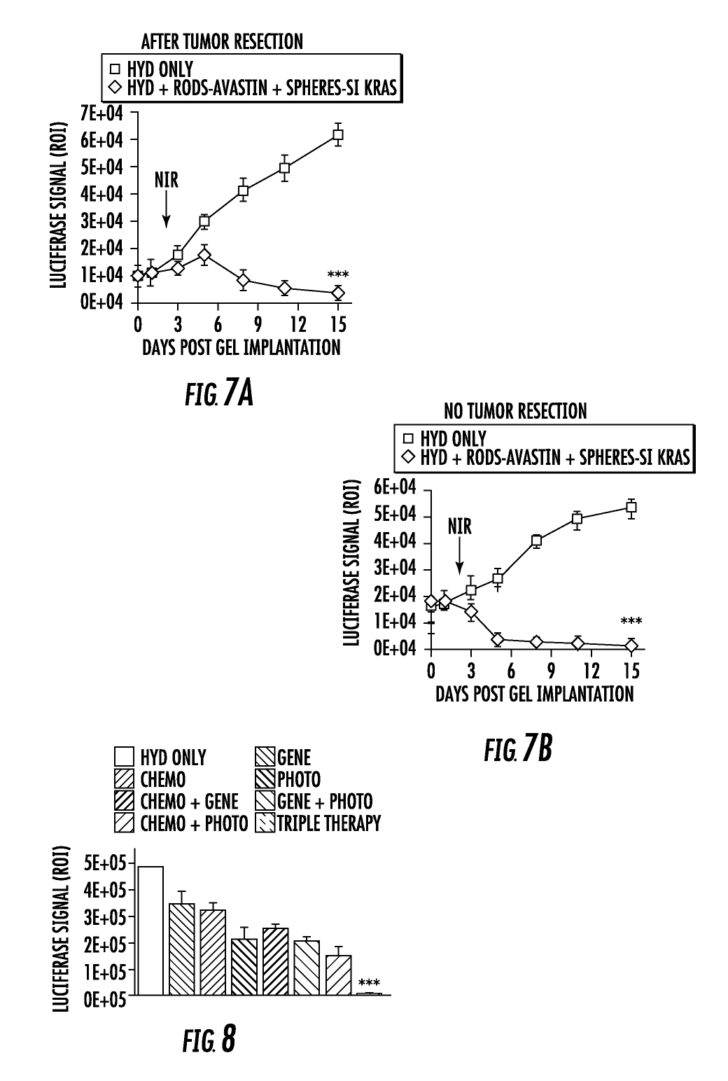

When the patch was used as a prophylactic measure following tumor resection, complete remission was achieved, as shown at FIG. 7A.

Even when the tumor was not resected, application of the hydrogel patch resulted in tumor abrogation, as shown at FIG. 7B, and as evidenced by bioluminescence imaging (more than 90%, n=5, P<0.001) 15 days post hydrogel implantation, thereby eliminating the need for resection. It was found that 40% of the tumors recurred following resection in the absence of further treatment (hydrogel only control group), while the triple therapy hydrogels implanted post resection were able to completely destroy the remaining cancer cells, thus preventing tumor re-growth (see FIG. 7A).

This effect was perpetual, because at 170 days post-tumor resection there was still no evidence of tumor recurrence in the triple-therapy hydrogel group. This result was determined by live imaging of the SCID hairless cogenic mice with colorectal tumor xenografts implanted with hydrogels only or embedded with drug gold nanorods and siRNA gold nanospheres with NIR treatment after tumor resection (n=5 per group), during 170 days.

When comparing the efficacy of single, dual or triple therapy (i.e. single therapy (chemo, gene, or photo-therapy), dual therapy (gene-chemo, chemo-photo, or gene-photo), or triple therapy (chemo-gene-photo)), the triple therapy was surprisingly more effective than the other therapies or combinations thereof, as shown at FIG. 8. The triple therapy appeared to have a synergistic effect on the tumors.

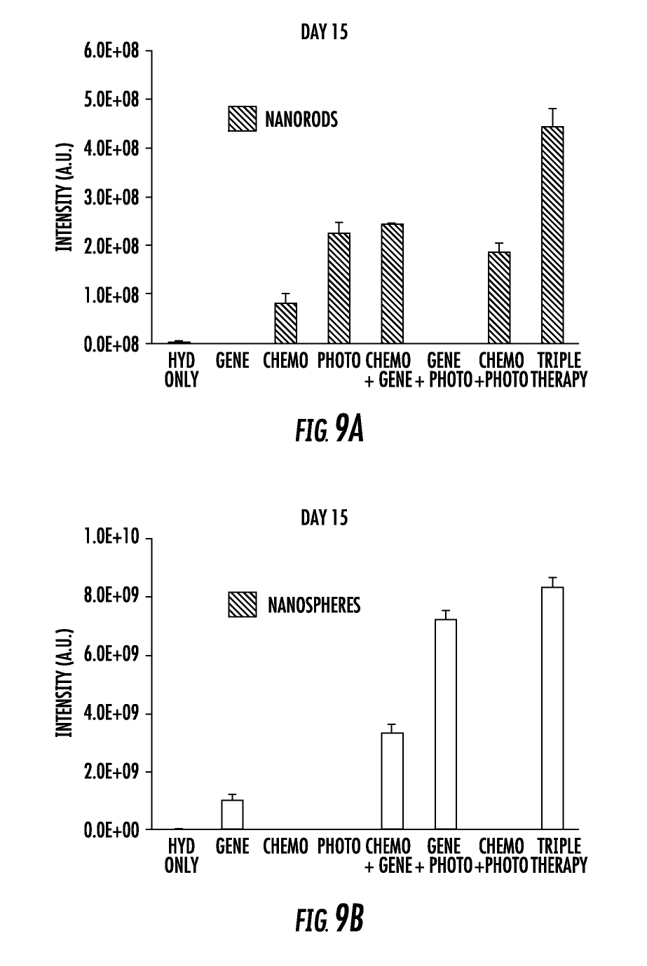

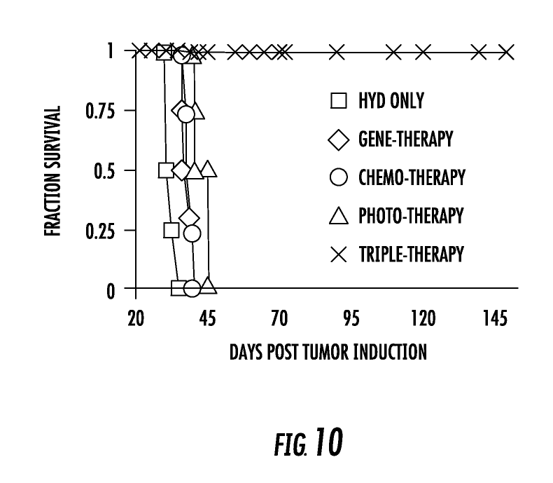

Irrespective of treatment modality, phototherapy add-on resulted in increased accumulation of nanoparticles in the tumor both for nanorods and for nanospheres, as quantified by their respective fluorescence signal in mouse tissues using a live imaging system 15 days after single, dual, or triple therapy, as depicted at FIG. 9A and FIG. 9B. The combination of the relatively instantaneous killing of cancer cells following phototherapy with the high accumulation of nanoparticles containing drug/gene may have resulted in a ‘higher effective dose’ of the cargo per cell, which further contributed to tumor abrogation.

To further demonstrate the effectiveness of the triple therapy local hydrogel patch, the efficacy of the local delivery was compared with systemic (tail-vein injection) or intratumoral administration of the nanoparticles alone, with drug-gold nanorods and/or siRNA-gold nanospheres at a final concentration of 10 nM each. Although the local administration of a hydrogel patch doped with a triple therapy combination resulted in more than 90% tumor shrinkage (see FIG. 8), only 60% or 40% tumor reduction was attained following intratumoral and systemic administration of the same therapy combination. Moreover, the intratumoral and system administration lead to poor survival increments (36 days post tumor induction for systemic therapy, 41 days for intratumoral injection and at least 150 days for the local hydrogel therapy). These results were obtained by Kaplan-Meier curves for the mice treated with triple therapy modality administered via local implantation of the hydrogel or injected via systemic and intratumoral administrations. Statistical analysis (n=5) was performed with a Log-Rank Mantel-Cox test (P=0.007). The survival cut-off criteria included tumor ulceration or compassionate euthanasia, aggregate tumor burden >1 cm in diameter, or if the tumor impeded eating, urination, defecation or ambulation. The systemic administration resulted in non-specific accumulation in the kidneys, spleen, and liver. These results provide convincing evidence that the hydrogel patch was instrumental to the achievement of superior therapeutic performance.

To validate the safety of the compositions, organs were harvested from the mice 15 days after hydrogel implantation, and H&E stained for histopathology analysis. H&E staining revealed that the in vivo application of a hydrogel (dendrimer and dextran only) or the triple therapy hydrogels did not cause any organ damage (i.e., lung, liver, kidney, spleen, heart, intestine), when compared to control group (without hydrogel treatment).

Nevertheless, the tumor tissue showed extensive reduction in vascularization, in accordance with tumor size reduction following treatment. No in vivo toxicity or other physiological complications were observed in all of the animals for 15 days post hydrogel exposure as indicated by the preservation of steady body weight, which indicated that the hydrogel compositions were biocompatible. Assessment of in vivo toxicity via mice body weight evaluation was performed on all the animal groups during 35 days after tumor induction and 15 days after hydrogel implantation.

A survival study was performed for the mice following triple therapy (gene, chemo and photo therapy combination) or control single therapies (gene-siRNA gold nanospheres, chemo-drug gold nanorods, or photo-gold nanorods). Mice survival following triple therapy showed a highly significant survival advantage (P=0.008, 100% survival for at least 170 days) compared with hydrogel only, or the single therapy groups, as shown at FIG. 10. FIG. 10 includes Kaplan-Meier curves for mice treated with hydrogel scaffolds for gene therapy (siRNA gold nanospheres), chemo-therapy (drug gold nanorods), phototherapy (gold nanorods), or triple therapy (gene, chemo and photo therapy combination). Statistical analysis (n=5) was performed with a Log-Rank Mantel-Cox test (P=0.008). Survival cut-off criteria included tumor ulceration or compassionate euthanasia, when aggregate tumor burden >1 cm in diameter, or if a tumor impeded eating, urination, defecation or ambulation.

Survival followed substantially the same trend seen with tumor size reduction following the different treatment modalities. This data was significant because patients having colorectal cancer typically will undergo surgery to resect the tumor and in more advanced stages undergo chemotherapy, but still carry the risk of recurrence owing to remaining cells at the tumor resection site that will continue to proliferate.

Immunohistochemical analysis showed that Ki67 expression, which is a cellular marker exclusively linked with cell proliferation, was significantly reduced following local triple therapy when compared to hydrogel only group. Histologically, both drug gold nanorods and siRNA gold nanospheres were able to accumulate efficiently throughout the tumor tissue over time. Histological sections of the tumors (n=5) were stained with hematoxylin and eosin and for immunohistochemical analysis the tumors (n=5) were stained with the antibody anti-Ki67 (Abcam ab15580, dilution 1:200). Quantification of fluorescence signal of the tumors from mice treated with hydrogel containing the triple therapy was performed overtime (6, 24, 48, 72 hours and 15 days). The total fluorescence of the nanoparticles in the tissue was assessed by the sum of all pixel intensities for each colour channel (red for the gold nanospheres (DY647 dye) and green for the gold nanorods (Alexa-Fluor® 555)). During the image analysis the photons that were collected at each pixel are converted into pixel intensities using the Image-J software (version 1.49). Therefore, the sum of all pixel intensities in a region was proportional to the amount of particles in that region.

Nanorods presented with more substantial accumulation early (peak at 6 hours, 92.4±2.3% accumulation) followed by the accumulation of nanospheres (peak at 72 hours, 99.2±2.7% accumulation). These findings corroborated the observed result that all nanoconjugates accumulated exclusively in the tumors. Since delivery of these potent effector molecules was preferentially achieved in tumoral cells, no more than minor damage to surrounding cells was observed.

### Example 11

### Tumor Genetic Profile

Following assessment of therapeutic efficacy, the tumor genetic profile of treated mice was investigated due to the fact that the gene expression profile of a cell determines its phenotype and its response to various factors, including drugs, irradiation, and/or gene modulators.

Total RNA from LoVo-6-Luc-1 colorectal cancer cells and respective tumors from SCID hairless congenic mice was extracted using RNeasy Plus Mini Kit (Qiagen) according to the manufacture's protocol. cDNA was produced using High-Capacity cDNA Reverse Transcription Kit (Applied Biosystems) using 500 ng of total RNA. qRT-PCR was performed with Taqman® probes FAM-MGB for Kras, VEGF, SFRP4, ANK1, CTNNA2, EPHA1 and DEFB119. GAPDH was used as a reference gene. The reactions were processed using Light Cycler 480 II Real-time PCR machine (Roche) using TaqMan® Gene Expression Master Mix (Applied Biosystems) under the following cycling steps: 2 min at 50° C. for UNG activation; 10 min at 95° C.; 40 cycles at 95° C. for 15 s; 60° C. for 60 s. At least three independent repeats for each experiment were carried out. Gene expression was determined as a difference in fold after normalizing to the housekeeping gene GAPDH.

Colorectal tumor gene expression profiles in the mice were generated using a GeneChip® PrimeView™ Human Gene expression array. Total RNA from colorectal tumors was extracted using RNeasy Plus Mini Kit (Qiagen) according to the manufacture's protocol. RNA integrity was checked using the AATI Fragment Analyzer. Samples for the arrays were prepared using the Nugen Applause 3′ Amp kit and were hybridized to Affymetrix GeneChip® PrimeView™ Human Gene Expression Array. For gene expression heat-maps, the average expression across 3 replicates in each condition basing on the probes with the maximum signal intensity per gene was calculated. Heat-maps were created using Genesis 1.7.6. Pearson correlations were analysed using R 2.15.3. Scatter plots were also made by R 2.15.3. Heat maps were created by Genesis 1.7.6 basing on Pearson correlation coefficients of each replicate per condition.

These arrays involved more than 530,000 probes housing more than 36,000 transcripts and variants, which reproduce more than 20,000 genes plotted through RefSeq or via UniGene annotation. For gene expression heat-maps, the average expression across three independent replicates in each condition basing on the probes with the maximum signal intensity per gene was calculated.

A clustergram of genes that were differentially expressed by application of hydrogel scaffolds for gene-therapy (siRNA gold nanospheres) or chemo-therapy (drug gold nanorods) or photo-therapy (gold nanorods) in mice was built. The data were analysed by unsupervised hierarchical clustering, which revealed that the three treatment groups had distinct gene expression profiles. Using a threefold change relative to the sham group (i.e. treatment with hydrogel (dendrimer and dextran only) as a benchmark for differential expression, numerous genes were derived from the different therapeutic modalities.

Responders (i.e. photo-therapy) were clustered separately from non-responders (gene- and chemo-therapy) and untreated samples (i.e. Sham). For the clustering of differentially expressed genes, a total of 20087 genes were analysed and an absolute log2 fold-change of ≥1 with adjusted p value of ≤0.05 to define the differentially expressed gene sets was used. For the correlation between the untreated group and the photo-therapy group, the number of differential genes was 2943, 959 for the gene-therapy groups, and 380 for the chemo-therapy group. The volcano plots for the different therapies were assembled by plotting on the y-axis the negative log of the p-value. Genes that were highly deregulated were further to the left and right sides, while highly significant changes appeared higher on the plot.

In order to identify the relationship between the four ordered sets of treatments (in this case, gene expression data for several different therapies and for the un-treated group—Sham) a Pearson correlation matrix was developed. This matrix indicated both how the four sets were related and the strength of that relationship. All replicates correlated very well (the diagonal of the heat map), and all different therapy samples showed a very high correlation (>0.95).

In order to evaluate which genes were up- or down-regulated in response to the different treatments, a set was created with log2 fold-change cut off of ≥2, and realized a hierarchical clustering of these differentially expressed gene sets by using a row-normalized data that was prepared using the broad GenePattern tool Pre-process Dataset.

This analysis revealed the distinctive gene expression fluctuations related to photo-therapy with several clusters of genes that were only suppressed or induced in these groups. For example, photo-therapy-treated tumors showed substantial suppression of a large cluster of genes that are involved in cytoskeleton remodelling and cell adhesion via integrin-mediated cell adhesion or involved in cell proliferation and differentiation such as MYH11 (myosin heavy chain). In contrast, a large cluster of genes was induced only in the photo-therapy-treated tumors. This cluster contained genes mainly involved in metabolism such as RRM2B (Ribonucleotide Reductase M2 B), or in immune response mechanisms such as Bcl-6 (B-Cell CLL/Lymphoma 6).

Concerning gene-therapy, the major differences were the presence of a large cluster that was repressed in response to treatment. This included genes mainly involved in protein folding, membrane trafficking, and signal transduction, such as ABCA1 (ATP-Binding Cassette, Sub-Family A).

The response to chemo-therapy was associated with the depletion of several genes related to metabolism such as ADH6 (Alcohol Dehydrogenase 6) or in cell adhesion and migration processes such as FN1 (Fibronectin).

Using an alternative clustering/filtering, the top 25 largest absolute fold change genes were selected for each of the three therapy groups, then summarized into a string. These results revealed that there were 59 genes that were top25 in at least 1 therapy group. Most importantly, this analysis revealed that several miRNAs (i.e. miR-17, miR-18A, miR-19A, miR-19B1, miR-20A and miR-92A1) were down-regulated in all therapy groups, whereas in the control group (Sham) they were all up-regulated. These miRNAs were part of the miR-17˜92 cluster, which encrypts for six individual miRNAs: miR-17, miR-18A, miR-19A, miR-20A, miR-19B1, and miR-92A1. The miR-17˜92 miRNA cluster is positioned on human chromosome 13q31.3, in a genomic region that is frequently activated in lymphomas and other types of cancer. The mature miRNAs encoded by this locus are highly expressed in cancer cells promoting cell proliferation and blocking apoptosis, and have key roles in cancer progression due to their capability of supressing the expression of numerous tumor-associated proteins.

Supervised Gene Ontology (GO) and detailed Kyoto Encyclopedia of Genes and Genomes (KEGG) pathway analysis was used to identify the molecular pathways and describe the biological processes of the transcript profiling data. Pathway analyses were performed using Gene Set Enrichment Analysis tools and were based on differentially expressed genes in each treatment group including photo-, gene-, and chemo-therapy comparing with sham controls. Differential expression was defined as multiple testing adjusted p-values that were smaller than or equal to 0.05 and fold change greater than or equal to 1.5 fold by probes with the maximum intensity in each gene. The enrichment results with canonical pathway gene sets C2CP were reported and false discovery rate (FDR) of 25% or less was used to select interesting gene sets for hypothesis generation.

The GO terms of the altered genes belonged to multiple pathways mainly related to metabolism, intracellular transport mechanisms, receptor signalling, cell cycle and apoptosis, immune and defence response and transcription/translation processes. These observations indicated that the features of the up-regulated and down-regulated genes resulting from the different therapies were absolutely distinct, especially following photo-therapy.

It was determined that in response to photo-therapy the molecular pathways involved were mainly regulated by genes controlling intracellular transport, such as membrane trafficking; whereas the mechanisms behind both the gene- and chemo-therapy approaches were mainly regulated by transcription (i.e., chromatin organization) and metabolism (i.e., metabolism of proteins, of amino acids, of nucleotides, of nitric oxide or of carbohydrates).

In order to identify key genes involved in colorectal cancer that could be altered in response to each therapy, MetaCore was used, which is an integrated software suited for functional analysis of microarrays, based on a high-quality, manually-curated database of signalling and metabolic pathways. The GO data was clustered by biomarkers in disease, via the calculation of the p-value distribution of diseases (biomarkers) for the differentially expressed genes in each treatment group. A Venn diagram showed that only photo-therapy alone altered more than 2,000 genes, with 14 genes as key players in colorectal cancer progression, the genes being mainly related with response to stress and to an external stimulus such as metabolism, receptor signalling and defence mechanisms like immune system pathways. For example, PLG (plasminogen), whose activation leads to decrease in apoptosis and tumor profession, is down-regulated following photo-therapy when compared to sham that is up-regulated.

Another example was the SFRP4 (Secreted Frizzled-Related Protein 4), which is associated with the Wnt signalling and was down-regulated in response to photo-therapy. It is well known that an abnormal regulation of the Wnt signalling pathway has a key role throughout the onset and progression of colorectal cancer, as this pathway is activated in colon cancer cells.

Another gene that was highly up-regulated in response to photo-therapy was the ANK1 (Ankyrin 1), which plays key roles in cytoskeleton remodelling and maintenance of specialized membrane domains.

A comparison of all the common genes and variants in the different treatment groups revealed that the majority of genes related to colorectal cancer progression were mainly associated with intracellular transport (e.g., CTNNA2, which is a structural constituent of cytoskeleton and cadherin binding and was highly repressed in the photo-therapy group only), receptor signalling (e.g., EPHA1, which is protein-tyrosine kinase that has been implicated in colorectal carcinogenesis and that was found to be slightly upregulated in the photo-therapy group), or immune system (e.g., DEFB119, which is a β-defensin and a diagnostic markersin colorectal cancer that was highly repressed in the photo-therapy group only). The qPCR validation of the expression levels of these genes was determined and plotted, and the results were in concordance with the microarray results and further confirmed the reliability and accuracy of the array chip approach.

The molecular pathways that determine the effectiveness of anti-tumor response following a specific therapy are poorly understood. Therefore, unravelling the molecular pathways involved in heat stress perception and signalling, as well as the one concerning drug delivery and gene therapy approaches, can be significant. The profiling analysis facilitated the identification of potential intermediaries of tumor response to therapy. Employing a network analysis methodology, numerous cores that were related to the response to therapy were classified and ranked. Specifically, the whole-gene expression analysis of the tumors from treated mice suggested that phototherapy treatment caused the repression and induction of higher number of genes and variants when compared to gene or chemo-therapy. Molecular pathways that are mainly regulated by genes controlling intracellular transport, such as membrane trafficking, were “turned on” in response to photo-therapy; whereas transcription and metabolism mechanisms were stimulated following both gene- and chemo-therapy. Moreover, the gene expression analysis revealed that the efficacy of the treatment was correlated with the number of genes altered; the photo-therapy group was highly efficacious in reducing tumor size and increasing mice survival, while inducing the highest number of altered genes.

### Example 12

### Canonical Pathway Kinetics in Local Triple-Therapy

Gene expression data analysis afforded insight into the contribution of each therapeutic modality to tumor abrogation. Nevertheless, the contribution of the combination of the three therapeutic modalities: gene-, chemo- and photo-therapy was a key element in studying the host response based on genome wide microarray analysis. However, 15 days after treatment with the triple therapy hydrogel, the RNA in the excised tumors was completely degraded following irradiation, and hence did not pass the quality check required for the microarray studies. Therefore, a series of in vivo experiments was conducted in which the gene expression alteration succeeding triple therapy over time was studied, which provided information about the canonical pathway kinetics of the triple therapy combination.

The tumor genetic profiles of mice treated with the triple therapy changed over time. This was evident upon comparing and contrasting the altered genes in treated mice at 6, 24, 48 or 72 hours. At the intersection of the genetic profiles of all the treatment groups there were more than 500 common genes. These genes were mainly related to apoptosis and survival (Beta-2 adrenergic receptor), as well as stress response (serotonin receptor HTR1A), immune system (MAPK signalling), chemotaxis (i.e., CCL16, CCL20, CXCL16 and CCL25), and regulation of extracellular matrix (matrix metalloproteinase (MMP) family).

Based on the GO analysis, the GO terms of the altered genes changed over time, and increased in number. At 6 hours, the majority of the altered genes belonged to pathways that were mainly related to metabolism, cell cycle, and apoptosis (down-regulation of caspase-10 and up-regulation of death receptors DR3, 4 and 5), and immune and defense responses (up-regulation of IL-22, IL-1 and IL-4). At 24 hours, there was a significant increase in altered genes related to metabolism (up-regulation of cytochromes CYPs and hydroxysteroid dehydrogenases HSDs) and transcription (up-regulation of histones H1, H2 and G-protein). At 48 and 72 hours the molecular pathways controlling the tumor cells shifted completely to processes related to receptor signalling (down-regulation of Ephrin receptors, EGFR and VEGF (due to the Avastin® action), and regulation of extracellular matrix (up-regulation of E-cadherin, plasmin, and MMPs, and down-regulation of collagen III and IV). From 6 h to 72 h the number of altered genes also changed from 209 to 3589, which correlated with the maximum therapeutic efficacy observed after 72 h using the local triple therapy combination. This demonstrated that the genetic profile was determined by the therapeutic modality.

Taken together, the wide genome array assays from tumors in response to each of the single therapeutic modalities and especially to the triple combination therapy provided potential biomarkers for gene/drug/photo-therapy in clinical application. The biomarkers identified herein via quantitative gene expression analysis may be used to better design other compositions that can target specific genes/pathways to achieve a desired clinical outcome based on prognosis analysis and correlations between pre-clinical studies and patient-derived samples in order to choose the optimal disease-associated biomarkers. A comprehensive scrutiny of the genes in the tumor microenvironment in response to therapy as the ones realized herein may further determine which ‘cargos’ a material should contain in light of the observed tumor microenvironment characteristics and response to therapy.

1. A composition comprising:
a first metal nanoparticle functionalized with a drug and a targeting biomolecule; and
a second metal nanoparticle functionalized with the targeting biomolecule and an siRNA;
wherein the targeting biomolecule is configured to bind to a unique or overexpressed biomarker of a diseased cell, and the siRNA is configured to silence a gene of the diseased cell.

2. The composition of claim 1, further comprising a hydrogel in which the first metal nanoparticle and the second metal nanoparticle are dispersed.

3. (canceled)

4. The composition of claim 1, wherein the second metal nanoparticle is further functionalized with a fusogenic peptide.

5. The composition of claim 4, wherein the fusogenic peptide is an HA1 peptide.

6. The composition of claim 4, wherein the fusogenic peptide is present in the composition at an amount of about 20 mols to about 40 mols per particle of the second metal nanoparticle.

7. (canceled)

8. The composition of claim 1, wherein the first metal nanoparticle is a nanorod.

9. The composition of claim 1, wherein the drug is an anti-angiogenic agent.

10. (canceled)

11. The composition of claim 1, wherein the drug is present in the composition at an amount of about 10 mols to about 20 mols per particle of the first metal nanoparticle.

12. (canceled)

13. The composition of claim 1, wherein the targeting biomolecule comprises a TCP-1 peptide.

14. The composition of claim 1, wherein the targeting biomolecule is present in the composition at an amount of about 20 mols to about 40 mols per particle of the first metal nanoparticle, and about 20 mols to about 40 mols per particle of the second metal nanoparticle.

15. (canceled)

16. The composition of claim 1, wherein the diseased cell is a tumor cell.

17. The composition of claim 1, wherein the siRNA comprises an anti-Kras siRNA.

18. The composition of claim 1, wherein the siRNA is present in the composition at an amount of about 90 mols to about 120 mols per particle of the second metal nanoparticle.

19. (canceled)

20. The composition of claim 1, further comprising a drug linker, wherein the drug linker is covalently bonded to the first metal nanoparticle and the drug.

21. The composition of claim 20, wherein the drug linker comprises (i) a sulfur atom covalently bonded to the first metal nanoparticle, and (ii) an ester moiety covalently bonded to the drug.

22. The composition of claim 1, further comprising a targeting biomolecule linker, wherein the targeting biomolecule linker is covalently bonded to the targeting biomolecule and at least one of the first metal nanoparticle and the second metal nanoparticle.

23. The composition of claim 22, wherein the targeting biomolecule linker comprises (i) a sulfur atom that is covalently bonded to at least one of the first metal nanoparticle and the second metal nanoparticle, and (ii) an ester moiety covalently bonded to the targeting biomolecule.

24. The composition of claim 1, wherein the first metal nanoparticle is a gold nanoparticle.

25. The composition of claim 24, wherein the gold nanoparticle is a gold nanorod.

26. (canceled)

27. The composition of claim 1, wherein the second metal nanoparticle is a gold nanoparticle.

28. The composition of claim 1, wherein the second metal nanoparticle is a gold nanosphere.

29. (canceled)

30. A method of drug delivery, the method comprising:
providing a first solution comprising a first polymer component comprising a first polymer having one or more aldehydes;
providing a second solution comprising at least one of (i) a dendrimer comprising at least two branches with one or more surface groups, wherein about 25% to 100% of the surface groups comprise at least one primary or secondary amine, and (ii) a second polymer component comprising a second polymer having one or more amines;
combining the first and second solutions together to produce a hydrogel composite; and
contacting one or more biological tissues with the hydrogel composite,
wherein at least one of the first solution and the second solution comprises the composition of claim 1, the composition being a treatment or prophylaxis composition.

31. The method of claim 30, further comprising irradiating the first metal nanoparticle with a light source, wherein the light source emits one or more wavelengths of light that are (i) converted to heat by the first metal nanoparticle, (ii) effective to release the drug from the first metal nanoparticle, or (iii) a combination thereof.

32-60. (canceled)

61. A kit for making a hydrogel composite, the kit comprising:
a first part that includes a first solution comprising a first polymer component comprising a first polymer having one or more aldehydes; and
a second part that includes a second solution comprising at least one of (i) a dendrimer comprising at least two branches with one or more surface groups, wherein about 25% to 100% of the surface groups comprise at least one primary or secondary amine, and (ii) a second polymer component comprising a second polymer having one or more amines,
wherein at least one of the first solution and the second solution comprises the composition of claim 1.

62. The kit of claim 61, further comprising a syringe, wherein the first solution and the second solution are stored in the syringe.

63-64. (canceled)

65. The kit of claim 61, further comprising a light source.

66. A method for local delivery of a drug to a biological tissue, comprising:
applying to the biological tissue the composition of claim 2; and
permitting at least one of the first metal nanoparticle and the second metal nanoparticle to diffuse from the composition into the biological tissue.

67. The method of claim 66, further comprising irradiating the first metal nanoparticle with a light source, wherein the light source emits one or more wavelengths of light effective to release the drug from the first metal nanoparticle.

68. A method of treatment or prophylaxis of cancer in a patient, comprising:
administering to the patient in need thereof an effective amount of the composition of claim 1; and
binding the targeting biomolecule to a cancer cell to permit the siRNA to silence a gene of the cancer cell.

69. The method of claim 68, further comprising irradiating the first metal nanoparticle with light effective to release the drug from the composition into the patient.

70. The method of claim 68, further comprising irradiating the first metal nanoparticle with light effective to heat the first metal nanoparticle to a temperature effective to kill or damage a cancer cell.

71. (canceled)
